(19) United States
(12) Patent Application Publication
(10) Pub. No.: US 2017/0216244 A1
(43) Pub. Date: Aug. 3, 2017

(54) METHODS FOR TREATING DIABETIC NEPHROPATHY

(71) Applicant: YALE UNIVERSITY, New haven, CT (US)

(72) Inventor: Alda TUFRO, Milford, CT (US)

(21) Appl. No.: 15/119,861

(22) PCT Filed: Mar. 4, 2015
(86) PCT No.: PCT/US15/18732

§ 371 Date: Aug. 18, 2016

Related U.S. Application Data
(60) Provisional application No. 61/948,774, filed on Mar. 6, 2014.

(51) Int. Cl. A61K 31/352; A61K 36/06; A61K 45/06

(52) U.S. Cl. CPC A61K 31/352; A61K 45/06; A61K 36/06

(57) ABSTRACT

The present invention provides methods for treating or preventing diabetic nephropathy and its related pathologies by inhibiting podocyte Semaphorin3a (Sema3a). The invention also provides a method for identifying or characterizing a compound useful for treating or preventing diabetic nephropathy.

### CROSS-REFERENCE TO RELATED APPLICATIONS

The present application claims priority under 35 U.S.C. §119(e) to U.S. Provisional Patent Application No. 61/948,774, filed Mar. 6, 2014, which application is incorporated herein by reference in its entirety.

### STATEMENT REGARDING FEDERALLY SPONSORED RESEARCH OR DEVELOPMENT

This invention was made with government support under Grants Nos. DK064187, DK098824 and DK079310 awarded by the National Institutes of Health. The government has certain rights in the invention.

### BACKGROUND OF THE INVENTION

Diabetic nephropathy is the major cause of end-stage renal disease worldwide. (Fineberg et al., Nat Rev Endocrino12013;9(12):713-723). Diabetic nephropathy affects approximately 30% of both type 1 and type 2 diabetic patients. The precise determinants of susceptibility to develop diabetic nephropathy are unknown, and the pathogenic molecular mechanisms leading to progression to renal failure are not fully understood (Forbes et al., Physiol Rev 2013; 93(1):137-188; Badal et al., Am J Kidney Dis 2014;63(2 Suppl 2):563-83).

The onset of diabetic nephropathy is highlighted by glomerular filtration barrier functional and morphologic abnormalities, namely microalbuminuria, hyperfiltration, glomerular basement membrane (GBM) thickening and glomerulomegaly (Forbes et al., Physiol Rev 2013; 93(1):137-188; Mogensen et al., Diabetes. 1983;32 Suppl 2:64-78). VEGF-A mediates some of these changes locally, modulated by reactive oxygen species, AGEs, angiotensin II, and low nitric oxide, which act in concert with the diabetic milieu (Tufro et al., Semin Nephrol 2012;32(4):385-393). Additional angiogenic factors, such as platelet-derived growth factor-B and angiopoietin 2 contribute to the development of proteinuria in diabetic mice (Gnudi, Nephrol Dial Transplant. 2012;27(7):2642-2649; Dessapt-Baradez et al., J Am Soc Nephrol. 2014;25(1):33-42; Suzuki et al., Diabetologia. 2011;54(11):2953-2962; Sung et al., J Am Soc Nephrol 2006;17(11):3093-3104; Nakagawa et al., Diabetes 2009;58(7):1471-1478). Semaphorin3a (Sema3a), a member of the Semaphorin family of guidance proteins, is characterized by its ability to collapse actin cytoskeleton and disassemble F-actin in multiple cell types (Tran et al., Annu Rev Cell Dev Biol 2007;23:263-292; Hung et al., Cytoskeleton (Hoboken) 2011;68(8):415-433). In the kidney, podocytes and ureteric bud-derived tubular cells synthesize Sema3a (Villegas et al., Mech Dev 2002;119 Suppl 1:S149-153). Sema3a is required for normal glomerular filtration barrier development and podocyte differentiation (Reidy et al., Development 2009;136(23):3979-3989), but Sema3a gain-of-function disrupts the slit-diaphragm, causes foot process effacement and proteinuria (Reidy et al., Am J Pathol 2013;183(4):1156-1168). Sema3a induces podocyte contraction and F-actin collapse cell autonomously. A membrane protein complex consisting of a binding receptor, neuropilinl, and a signaling receptor, plexinA₁, mediates Sema3a signals (Kolodkin et al., Cell 1997;90:753-762; Takahashi et al., Cell 1999;99(1):59-69). Neuropilinl (NRP1) is also a co-receptor for VEGF-A, and both ligands compete for neuropilinl binding (Miao et al., J Cell Biol 1999;146(1):233-242). PlexinA₁ intracellular signaling involves several pathways to regulate cell shape and cytoskeleton, including integrins, MICAL (molecule interacting with CasL), CRMP (collapsing response mediator protein) and small GTPases, as well as interactions with receptor tyrosine kinases and other membrane proteins. In podocytes, plexinAᵢ interacts with nephrin directly. MICALs are cytoplasmic flavoxygenase proteins that regulate cell shape, migration and exocytosis through a redox-dependent mechanism (Terman et al., Cell 2002; 109(7):887-900). MICALs bind plexinA receptors directly, and induce F-actin loss by decreasing actin polymerization, bundling and branching (Hung et al., Science 2011;334(6063):1710-1713; Hung et al., Nat Cell Biol 2013;15(12):1445-1454), thereby linking extracellular semaphorin signals to actin dynamics and the cytoskeleton. Sema3a upregulation has been observed in diabetic mouse kidneys (Veron et al., Diabetologia 2011;54(5):1227-1241), but Sema3a pathophysiologic function in diabetic nephropathy remains unknown.

There is a critical need for the identification of novel pathogenic factors and targetable signaling pathways that mediate diabetic nephropathy. The identification of novel therapeutic targets for diabetic nephropathy is essential to the development of new therapies for treatment of diabetic nephropathy and improvement of disease outcome (Breyer, Semin Nephrol 2012;32(5):445-451). The present invention addresses this need.

### SUMMARY OF THE INVENTION

The invention includes a method for treating or preventing diabetic nephropathy in a mammal in need thereof, the method comprising administering to the mammal a therapeutically effective amount of an agent that inhibits podocyte Semaphorin3a (Sema3a).

The invention includes a method for reducing renal insufficiency in a mammal having or at risk for having diabetic nephropathy, the method comprising administering to the mammal a therapeutically effective amount of an agent that inhibits podocyte Semaphorin3a (Sema3a).

The invention also includes a method for improving kidney function in a mammal having or at risk for having impaired kidney function, the method comprising administering to the mammal a therapeutically effective amount of an agent that inhibits podocyte Semaphorin3a (Sema3a).

The invention further includes a method for reducing podocytopenia in a mammal having or at risk for having diabetic nephropathy, the method comprising administering to the mammal a therapeutically effective amount of an agent that inhibits podocyte Semaphorin3a (Sema3a).

The invention further includes a method for reducing glomerulosclerosis or glomerular filtration rate in a mammal having or at risk for having diabetic nephropathy, the method comprising administering to the mammal a therapeutically effective amount of an agent that inhibits podocyte Semaphorin3a (Sema3a).

The invention further includes a method for reducing proteinuria in a mammal having or at risk for having diabetic nephropathy, the method comprising administering to the mammal a therapeutically effective amount of an agent that inhibits podocyte Semaphorin3a (Sema3a).

The invention further includes a method for reducing albuminuria or correcting hypoalbuminemia in a mammal having or at risk for having diabetic nephropathy, the method comprising administering to the mammal a therapeutically effective amount of an agent that inhibits podocyte Semaphorin3a (Sema3a).

The invention further includes a method for identifying or characterizing a compound useful for treating or preventing diabetic nephropathy in a mammal in need thereof. The method comprises measuring the level of expression or activity of at least one of podocyte Semaphorin3a (Sema3a) nucleic acid, Sema3a encoded polypeptide, Sema3a receptor and any combination thereof, in a mammal treated with the compound and in an otherwise identical mammal not treated with the compound, wherein when the level of expression or activity of at least one of podocyte Sema3a nucleic acid, Sema3a encoded polypeptide, Sema3a receptor and any combination thereof, is decreased in the treated mammal as compared with the untreated mammal, the compound is useful for treating or preventing diabetic nephropathy.

In some embodiments, the mammal has a type 1 or a type 2 diabetes. In some embodiments, the mammal is a human. In certain embodiments, the administration route is parenteral. In other embodiments, the administration route is subcutaneous. In yet other embodiments, the administration comprises at least one from the group consisting of at least one administration for at least 2 days, at least one administration for at least 20 days, at least one administration for at least 30 days, a continuous administration for at least 2 days, a continuous administration for at least 20 days and a continuous administration for at least 30 days. In certain embodiments, the inhibiting agent comprises a specific Sema3a competitive binding inhibitor. In certain embodiments, the inhibitor comprises at least one from the group consisting of a natural product, a natural fungal product and a synthetic product. In some embodiments, the inhibitor is xanthofulvin. In other embodiments, the administration further comprises administering to the mammal an additional anti-diabetic therapeutic agent. In other embodiments, the agent that inhibits podocyte Semaphorin3a (Sema3a) and the additional anti-diabetic therapeutic agent are co-administered to the mammal. In yet other embodiments, the agent that inhibits podocyte Semaphorin3a (Sema3a) is in a pharmaceutical acceptable carrier.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIGS. 1A-1J are series of images and histograms depicting that podocyte SEMA3A is increased in human diabetic nephropathy. Dual fluorescent immunohistochemistry with SEMA3A and NPH2 antibodies was performed on frozen sections from human renal biopsies. FIGS. 1A-1C: Representative images from a biopsy with Class IV diabetic nephropathy exhibit strong SEMA3A signal (FIG. 1A) localized to podocytes, as indicated by co-localization with podocin (B) shown in merge (FIG. 1C). FIGS. 1D-1F: Representative images from a non-diabetic kidney biopsy (patient with hypertension and no evidence of renal pathology) exhibits minimal SEMA3A signal localized to podocytes. FIG. 1G: Representative low magnification image from non-diabetic kidney biopsy (MCD) demonstrates minimal SEMA3A in three glomeruli, while podocin expression is intact. FIG. 1H: Representative low magnification image from diabetic kidney biopsy demonstrates strong glomerular SEMA3A expression and variable co-localized podocin expression. Scale bars=50 μm (FIGS. 1A-1F) and 200 μm (FIGS. 1G-1H). FIGS. 1I-1J: Quantitation of IF signals establishes that SEMA3A increases ˜two fold in diabetic glomeruli (dark grey bars) as compared with non-diabetic glomeruli (empty bars), while podocin (light grey bars) does not change or decreases slightly. Data are expressed as mean±SEM IF integrated density/μm² (FIG. 1I) and IF % glomerular area (FIG. 1J). All glomeruli present in the biopsies were included in this quantitative analysis.

FIGS. 2A-2J is a series of graphs demonstrating that an excess of Sema3a in diabetic mice causes massive proteinuria and renal failure. FIG. 2A: Sema3a⁺ gain-of function and diabetes have additive effects increasing plasma Sema3a level. FIG. 2B: Diabetes increases Sema3a excretion and Sema3a⁺ gain-of function induces a synergistic increase. FIG. 2C: Quantification of albuminuria by ELISA (notice logarithmic scale): Sema3a⁺ gain-of-function in diabetic mice causes massive albuminuria, ˜40-fold higher than control diabetics. FIG. 2D: Coomassie blue stain of urine resolved by SDS-PAGE illustrates nephrotic range proteinuria in diabetic mice with Sema3a⁺ gain-of-function (DM-Sema3a⁺+dox). FIG. 2E: Diabetic mice with Sema3a⁺ gain-of-function develop hypoalbuminemia. FIGS. 2F-2H: Sema3a⁺ gain-of-function in diabetic mice induces renal failure: FIG. 2F: Sema3a⁺ gain-of-function induces doubling of plasma creatinine in diabetic mice, and a lesser increase in non-diabetic mice. FIG. 2G: Creatinine clearance decreases ˜60% in diabetic mice with Sema3a⁺ gain-of-function. FIG. 2H: Creatinine clearance correlates inversely with plasma Sema3a concentration. FIG. 2I: Albuminuria correlates directly with urine Sema3a excretion. FIG. 2J: Bar graphs illustrate body weight, kidney weight, urine output and blood glucose in all experimental groups. Notice severe polyuria in Sema3a⁺ gain-of-function diabetic as compared with control diabetic mice, without significant change in random blood glucose. FIGS. 2A-2J: Asterisk (*) indicates p<0.05 vs. corresponding control, numeral (#) indicates p<0.05 vs. non-diabetic control.

FIGS. 3A-3N: Sema3a⁺ gain-of-function in diabetic mice causes advanced diabetic nephropathy. FIGS. 3A-3B: PAS stain of non-diabetic kidneys (Sema3a⁺), (A) normal histology in control kidney (−dox), FIG. 3B: kidney from mouse receiving doxycycline (+dox) shows mesangial expansion. FIG. 3C: PAS from control diabetic kidney (DM-Sema3a⁺−dox) demonstrates mild mesangial expansion. FIG. 3D: Quantification of glomerular area indicates that Sema3a⁺ gain-of-function in diabetic mice induces glomerulomegaly, DM-Sema3a⁺+dox vs. DM-Sema3a⁺−dox, n=4 kidneys each, n=34±2 glomeruli/kidney. FIGS. 3E-3L: PAS and Jones' silver stains of Sema3a ⁺ gain-of-function diabetic kidneys (DM-Sema3a⁺+dox) show nodular Kimmelstiel-Wilson-like glomerulo-sclerosis (white arrows), mesangiolysis (black long arrows), diffuse glomerulosclerosis (white long arrow), fibrin caps (FIG. 3G and FIG. 3J thick black small arrows), arteriolar hyalinosis (FIG. 3L thick white small arrows), foam cells (FIG. 3M black stars), protein casts (FIGS. 3L-3M black asterisks), and interstitial infiltrates (FIG. 3M white cross). FIG. 3N: Quantification of glomerular nodules and mesangiolysis, shown as percentage of glomeruli with nodules or mesangiolysis/kidney in diabetic Sema3a⁺ gain-of-function (+dox) vs. diabetic control mice (−dox); n=134±6 and n=121±5 glomeruli/kidney were counted in n=5 and n=4 kidneys, respectively, p<0.05. Semiquantitative pathology score establishes significantly increased mesangial sclerosis in all Sema3a⁺ gain-function kidneys, whereas interstitial fibrosis occurs exclusively in Sema3a⁺gain-function diabetic mice (black bars). Scale bars=50μm (FIGS. 3A-3C and FIGS. 3E-3L). FIG. 3D and FIG. 3N: Asterisks in bar graphs indicate p<0.05, Sema3a⁺gain-of-function vs. diabetic control mice.

FIGS. 4A-4G are a series of images and histograms demonstrating that a Sema3a⁺ gain-of-function in diabetic mice causes diffuse foot process effacement (FPE), mesangial sclerosis and endothelial injury. FIG. 4A: TEM shows focal FPE, thick GBM in control diabetic glomeruli. FIG. 4B: Sema3a⁺ gain-of-function diabetic glomeruli TEM show diffuse FPE, podocyte vacuolization and absence of SD, GBM thickening, mesangial sclerosis and endothelial injury: EC swelling, and expansion of subendothelial space. FIG. 4C: Non-diabetic control glomeruli TEM show normal glomerular ultrastructure. FIG. 4D: Non-diabetic Sema3a⁺ gain-of-function glomeruli TEM shows focal FPE, mild endothelial swelling and mesangial sclerosis. FIG. 4E: Sema3a⁺ gain-of-function diabetic glomerulus shows complete FP effacement with collapsed F-actin (darker grey), thick GBM, mesangial matrix interposition (thin arrows), extensive mesangial matrix accumulation with electron dense fibrillar material (thick arrows), and narrow capillary lumen (cap). FIG. 4F: Quantitation of GBM thickness shows that excess Sema3a exacerbates GBM thickening in diabetic mice (black bar vs. empty bar) whereas it does not alter GBM in non-diabetic mice (light grey bars). FIG. 4G: Quantitation of FP width shows mild FPE in control diabetics (empty bar) and non-diabetics with excess Sema3a (dark grey bar), and massive FPE (˜3 fold vs. control DM) in Sema3a⁺ gain-of-function diabetics (black bar). Scale bars=2 μm.

FIGS. 5A-5H are a series of images and graphs depicting that laminin and collagen IV are increased in DM-Sema3a⁺ glomerular nodules. FIG. 5A: Dual-fluorescent IHC shows increased laminin and collagen IV co-localized to glomeruli from diabetic Sema3a⁺ gain-of-function mice (DM-Sema3a⁺+dox). FIGS. 5B-5C: Quantitation of immunoreactive laminin (FIG. 5B) and collagen IV (FIG. 5C) signals demonstrates significant increase in glomeruli from diabetic Sema3a⁺ gain-of-function mice. FIG. 5D: Western blot shows decreased total kidney laminin in diabetic Sema3a⁺ gain-of-function kidneys. FIG. 5E: Metalloproteinases 2 and 9 (MMP2 and MMP9) are downregulated in Sema3a⁺ gain-of-function diabetic kidneys. FIGS. 5F-5H: Renal VEGF-A signaling is not upregulated in diabetic mice with Sema3a⁺ gain-of-function. FIG. 5F: Plasma VEGF-A is elevated in all diabetic mice, as compared with non-diabetic mice, irrespective of Sema3a⁺ gain-of-function induction. FIG. 5G: Kidney VEGF-A protein expression measured by ELISA is downregulated in diabetic mice with Sema3a⁺ gain-of-function. FIG. 5H: VEGFR2 kidney protein expression is decreased in diabetic mice with Sema3a⁺ gain-of-function. Asterisk (*) indicates p<0.05 vs. corresponding control, numeral (#) indicates p<0.05 vs. non-diabetic control.

FIGS. 6A-6H are a series of images and graphs demonstrating that combined nephrin, WT-1 and αvβ3 integrin downregulation accentuates podocytopenia in diabetic Sema3a⁺ gain-of-function mice. Nephrin downregulation is shown by immunofluorescence (IF) (FIG. 6A) and western blot (FIG. 6B) in all diabetic mice and in non-diabetic mice with excess Sema3a, IF shows the least immoreactive nephrin in Sema3a⁺ gain-of-function diabetic glomeruli (FIG. 6A and FIG. 6E). Podocin immunoblots and IF are not significantly changed by diabetic nephropathy or excess Sema3a (FIG. 6B and FIG. 6G), except in severely damaged glomeruli (FIG. 6D, DM-Sema3a⁺+dox). FIG. 6B: WT1 downregulation shown by immunoblot, and IF WT1⁺ podocyte counts (FIG. 6C) in diabetic Sema3a⁺ gain-of-function kidneys (black bar) indicating podocytopenia; −dox and +dox non-diabetic WT1⁺ podocyte counts were not different and were pooled (grey bar). FIG. 6D: dual-IF shows αvβ3 integrin downregulation in diabetic Sema3a⁺ gain-of-function glomeruli. FIGS. 6E-6G: Quantitation of glomerular IF signals for nephrin, αvβ3 integrin and podocin. FIG. 6H: TEM from Sema3a⁺ gain-of-function diabetic glomerulus shows an open capillary loop with GBM ‘denuded’ of podocytes, illustrating the severe podocytopenia assessed by low podocyte (WT1⁺) counts shown in FIG. 6C. Asterisk (*) indicates p<0.05 vs. corresponding control, numeral (#) indicates p<0.05 vs. non-diabetic control.

FIGS. 7A-7E are a series of images and graphs showing that Sema3a signals in podocytes are mediated by MICALl. FIG. 7A: Western blots show that the Sema3a signaling pathway is expressed in the kidney. PlexinA₁, MICAL1 and β3 integrin are downregulated in Sema3a⁺ gain-of-function diabetic mice (black bars). Quantitation by densitometry is shown in adjacent bar graphs. Data are expressed as mean±SEM from ≧3 independent experiments. FIG. 7B: MICAL1 is expressed in cultured podocytes, not altered by 4 h exposure to high glucose. FIG. 7C: Co-immunoprecipitation demonstrates endogenous plexinA₁-MICAL1 interaction in podocytes, actin co-precipitates with the plexinA₁-MICAL1 complex; RS=rabbit serum control; HEK=whole cell lysate from HEK cells transiently transfected with full length MICAL1 was used as a control. FIG. 7D: Immunoblot shows MICAL1 knockdown by siRNA of ˜75%, confirmed by densitometric analysis. FIG. 7E: MICAL1 knockdown prevents Sema3a-induced podocyte contraction and F-actin collapse, assessed by rhodamine phalloidin staining. Data from ≧3 independent experiments are shown. Asterisk (*) indicates p<0.05 vs. corresponding control, numeral (#) indicates p<0.05 vs. non-diabetic control.

FIGS. 8A-8F are a series of images and graphs depicting that Sema3a inhibitor xanthofulvin ameliorates Sema3a-induced diabetic nephropathy phenotype in vivo and prevented podocyte contraction in vitro. FIG. 8A: Pre-treatment with 0.1 μM μM xanthofulvin for 60 min abrogates Sema3a-induced podocyte F-actin collapse, shape change and contraction, assessed by rhodamine phalloidin staining and cell area morphometric analysis (FIG. 8B); scale bars=20 μm. (FIGS. 8C-8E) Xanthofulvin constant subcutaneous infusion for 30 days (weeks 8-12 after diabetes onset) to Sema3a⁺ gain-of-function diabetic mice (DM-Sema3a⁺+dox+xanthofulvin) resulted in improved albuminuria, normalized plasma albumin and creatinine clearance (FIG. 8C, grey bars), mild mesangial hypercellularity and extracellular matrix expansion (FIG. 8D, right side panels) similar to control diabetic mice, a dramatic improvement from the diabetic nephropathy phenotype of Sema3a⁺ gain-of-function diabetic mice (DM-Sema3a⁺+dox, left side panels). Scale bars=50 μm (top panels) and 100 μm (bottom panels). FIG. 8E: TEM: (left panel) shows thick GBM and complete FPE in Sema3a⁺ gain-of-function diabetic glomerulus (+dox), (right panel) shows focal FPE with quite normal GBM in glomerulus from Sema3a⁺ gain-of-function diabetic mouse receiving xanthofulvin infusion (+dox+xanthofulvin). Scale bars=2 μm. FIG. 8F: Morphometry confirmed the improvement of GBM and FP width (grey bars). Asterisk (*) indicates p<0.05 vs. Sema3a⁺ gain-of-function diabetic mice.

FIGS. 9A-9I are a series of images and histograms demonstrating that the deletion of podocyte plexinAl attenuates diabetic nephropathy in mice. FIGS. 9A-9D: PAS and Jones' silver stains show severe diabetic nodular glomerulosclerosis in Sema3a⁺ gain-of function kidneys (A/C) and mild mesangial expansion and otherwise normal histology in diabetic Sema3a⁺:plexinA1^{pod} kidneys (FIG. 9B and FIG. 9D), scale bars=50 μm. FIGS. 9E-9F: TEM shows complete FP effacement, thickened GBM and endothelial swelling in Sema3a⁺ gain-of function diabetic glomeruli (FIG. 9E), whereas Sema3a⁺:plexinA1^{pod} (FIG. 9F) show very mild GBM thickening and virtually no FP effacement, as confirmed by morphometric analysis (I, n=4/group), scale bars=2 μm. FIGS. 9G-9H: Deletion of podocyte plexinA1 in diabetic mice results in mild albuminuria and normal creatinine clearance (grey bars) similar to wild type diabetic mice (empty bars), while Sema3a⁺ gain-of-function causes massive albuminuria and renal insufficiency (black bars). Asterisks (*) indicate p<0.05 vs. Sema3a⁺ gain-of-function.

FIG. 10 is a series of images illustrating that podocyte Sema3a expression is increased in diabetic mice. IHC: immunoreactive Sema3a and podocin co-localization indicates that Sema3a is expressed in podocytes. Clearly, Sema3a expression is increased in glomeruli from diabetic mice with Sema3a gain-of-function (bottom panel), as compared to control diabetic mice (top panel), whereas podocin expression is not different.

FIGS. 11A-11B are a series of histograms demonstrating that blood glucose (FIG. 11A) and body weight (FIG. 11B) at the completion of the study period are similar in all diabetic experimental groups. Data are expressed as mean+SEM, n.s.=not significantly different.

### DETAILED DESCRIPTION

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

By “alteration” is meant a change (increase or decrease) in the expression levels or activity of a marker or clinical indicator as detected by standard art known methods such as those described herein. As used herein, an alteration includes a 10%-100% change in measured levels (e.g., 10, 20, 30, 40, 50, 60, 75, 80, 85, 90, 95, 100%).

In this disclosure, “comprises,” “comprising,” “containing” and “having” and the like can have the meaning ascribed to them in U.S. Patent law and can mean “ includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

“Detect” refers to identifying the presence, absence or amount of the analyte to be detected.

As used herein, the term “diabetes” includes both insulin-dependent diabetes mellitus (i.e., IDDM, also known as type 1 diabetes) and non-insulin-dependent diabetes mellitus (i.e., NIDDM, also known as Type 2 diabetes). Type 1 diabetes, or insulin-dependent diabetes, is the result of an absolute deficiency of insulin, the hormone which regulates glucose utilization. Type II diabetes, or insulin-independent diabetes (i.e., non-insulin-dependent diabetes mellitus), often occurs in the face of normal, or even elevated levels of insulin and appears to be the result of the inability of tissues to respond appropriately to insulin. Most of the Type 2 diabetics are also obese. The World Health Organization defines the diagnostic value of fasting plasma glucose concentration to 7.0 mmol/l (126 mg/dl) and above for Diabetes Mellitus (whole blood 6.1 mmol/l or 110 mg/dl), or 2-hour glucose level of 11.1 mmol/L or more (200 mg/dL or more). Other values suggestive of or indicating high risk for diabetes mellitus include elevated arterial pressure of 140/90 mm Hg or more; elevated plasma triglycerides (1.7 mmol/L or 150 mg/dL or more) and/or low HDL-cholesterol (<0.9 mmol/L, 35 mg/dl for men; <1.0 mmol/L, 39 mg/dL women); central obesity (males: waist to hip ratio >0.90; females: waist to hip ratio >0.85) and/or body mass index exceeding 30 kg/m²; microalbuminuria, where the urinary albumin excretion rate is 20 ug/min or albumin:creatinine ratio 30 mg/g). Type 1 Diabetes can also be distinguished from type 2 Diabetes using a C-peptide assay, which is a measure of endogenous insulin production. The presence of anti-islet antibodies (to Glutamic Acid Decarboxylase, Insulinoma Associated Peptide-2 or insulin), or lack of insulin resistance, determined by a glucose tolerance test, is also indicative of type 1, as many type 2 diabetics continue to produce insulin internally, and all have some degree of insulin resistance.

As used herein the term “diabetic nephropathy” refers to a progressive kidney disease caused by angiopathy of capillaries in the kidney glomeruli (glomeruloscerosis). Diabetic nephropathy is also known as Kimmelstiel-Wilson syndrome, or nodular diabetic glomerulosclerosis and intercapillary glomerulonephritis, it is characterized by nephrotic syndrome and diffuse glomerulosclerosis. Diabetic nephropathy clinical syndromes include but are not limited to persistent albuminuria (>300 mg/d or >200 μg/min usually confirmed on at least 2 occasions 3-6 months apart), progressive decline in the glomerular filtration rate (GFR) and elevated arterial blood pressure. Progression of diabetic nephropathy is characterized by a fairly predictable pattern of events. Generally, the time course of development of diabetic nephropathy is as follows. Glomerular hyperfiltration and renal hypertrophy occur in the first years after the onset of diabetes and are reflected by increased glomerular filtration rate (e.g., from a normal glomerular filtration rate of about 120 ml/min to about 150 ml/min in humans). During the first 5 years of diabetes, pathological changes, such as glomerular hypertrophy, thickening of the glomerular basement membrane, and glomerular mesangial volume expansion, are observed. Glomerular filtration rate gradually returns to normal. After 5 to 10 years of diabetes, individuals begin to excrete albumin/proteins in the urine (albuminuria or proteinuria). The basement membrane thickening and glomerular volume expansion seen in early stages of the disease can accumulate in late stage diabetic nephropathy, leading to obliteration of the capillary lumen, and, eventually, to glomerulosclerosis. Once overt diabetic nephropathy is present, a steady decline in the glomerular filtration rate occurs, and approximately half of individuals reach end-stage renal disease in 7 to 10 years. Clinically, the stages of development and progression of diabetic nephropathy in humans have been well described. Stage I diabetic nephropathy is associated with increased kidney (i.e., glormerular) filtration (i.e., hyperfiltration, resulting from increased blood flow through the kidneys and glomeruli), increased glomerular filtration rate, glomerular hypertrophy, and enlarged kidneys. Stage II diabetic nephropathy is a clinically silent phase associated with continued hyperfiltration and kidney hypertrophy. Thickening of the glomerular basement membrane and mesangial expansion occurs. Stage III diabetic nephropathy (also known as incipient diabetic nephropathy) is associated with micro albuminuria and micro proteinuria. Micro albuminuria is defined as 30 to 300 mg/day urinary albumin in a 24-hour collection, 20-200 μg/min urinary albumin, or 30 to 300 μg/mg creatinine in a spot collection. The kidneys progressively lose the ability to filter waste and blood levels of creatinine and urea-nitrogen increase. Glomerular basement membrane thickening and mesangial expansion continue to occur with increasing severity. Stage IV diabetic nephropathy (also known as overt diabetic nephropathy) is associated with macro albuminuria (i.e., clinical albuminuria) and creatinine and blood urea-nitrogen (BUN) levels in the blood continue to rise. Macro albuminuria is defined as greater than 300 mg/day urinary albumin in a 24-hour collection, greater than 200 μg/min urinary albumin, or greater than 300 μg/mg creatinine spot collection. Once overt diabetic nephropathy occurs, glomerular filtration rate gradually falls over a period of several years. Stage V diabetic nephropathy occurs with end-stage renal disease and kidney failure. “Diagnostic” means identifying the presence or nature of a pathologic condition. While a particular diagnostic method may not provide a definitive diagnosis of a condition, it suffices if the method provides a positive indication that aids in diagnosis.

The expression “difference in the level of” or “differentially present” refers to differences in the quantity and/or the frequency of a marker present in a sample taken from subjects having a disease as compared to a control subject. A marker can be differentially present in terms of quantity, frequency or both. A difference in the level of a polypeptide is present between two samples if the amount of the polypeptide in one sample is statistically significantly different from the amount of the polypeptide in the other sample. Alternatively or additionally, a polypeptide is differentially present between two sets of samples if the frequency of detecting the polypeptide in a diseased subjects' samples is statistically significantly higher or lower than in the control samples. A marker that is present in one sample, but undetectable in another sample is differentially present.

A “disease” is a state of health of an animal wherein the animal cannot maintain homeostasis, and wherein if the disease is not ameliorated then the animal's health continues to deteriorate. Examples of diseases include a metabolic syndrome or metabolic-syndrome related diseases, including but not limited to obesity, diabetes, including type I and type II diabetes, insulin-deficiency, insulin-resistance, insulin-resistance related disorders, glucose intolerance, syndrome X, inflammatory and immune disorders, dyslipidemia, metabolic syndrome, non-alcoholic fatty liver, abnormal lipid metabolism, sleep apnea, hypertension, high cholesterol, atherogenic dyslipidemia, hyperlipidemic conditions such as atherosclerosis, hypercholesterolemia, and other coronary artery diseases in mammals, and other disorders of metabolism. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

“Effective amount” or “therapeutically effective amount” are used interchangeably herein, and refer to an amount of a compound, formulation, material, or composition, as described herein effective to achieve a particular biological result or provides a therapeutic or prophylactic benefit. Such results may include, but are not limited to, anti-tumor activity as determined by any means suitable in the art.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein “endogenous” refers to any material from or produced inside an organism, cell, tissue or system.

As used herein, the term “exogenous” refers to any material introduced from or produced outside an organism, cell, tissue or system.

The term “expression” as used herein is defined as the transcription and/or translation of a particular nucleotide sequence driven by its promoter.

As used herein the term “glomerulosclerosis” refers to scarring or hardening of the kidneys' blood vessels located in the kidneys (glomeruli). The glomeruli filter the blood as it passes through the kidneys and their alteration cause an abnormal amount of protein in the urine (proteinuria). Glomerular filtration rate is a measurement of the volume of filtrate made by the kidneys per minute. Measurement of glomerular filtration rate in human subjects has been accepted as the best overall index of kidney function in health and disease. (Smith, Diseases of the kidney and urinary tract, In: Structure and Function in Health and Disease, New York; Oxford Univ. Press, 1951:836-887.) Glomerular filtration rate can be determined by various methods, such as by measuring the urinary clearance of a filtration marker, such as inulin, iothalamate, or iohexol. More commonly, glomerular filtration rate is estimated by determining clearance of creatinine, a protein produced by muscle and released into the blood. Creatinine clearance (often expressed as ml/min) can be determined by comparing the level of creatinine collected in urine over a given period of time, e.g., 12 or 24 hours, with the creatinine level in blood. A typical creatinine clearance rate is about 97 to 137 ml/min in adult males, and about 88 to 128 ml/min in adult females. In clinical practice, creatinine clearance is most often estimated from the serum creatinine concentration. Creatinine clearance is related directly to the urine creatinine excretion and inversely to serum creatinine concentration. Various formulas that provide estimates of creatinine clearance, and therefore estimates of glomerular filtration rate, using parameters such as serum creatinine concentration, age, sex, and body size, have been developed and are standard in the art (Cockcroft and Gault Nephron 1976, 16:3141; Levey et al Annals of Internal Medicine, 1999,130:462-470; Rule et al., Ann Intern Med, 2004, 141:929-937). “Homologous” as used herein, refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

“Identity” as used herein refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an Arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

The term “immune response” as used herein is defined as a cellular response to an antigen that occurs when lymphocytes identify antigenic molecules as foreign and induce the formation of antibodies and/or activate lymphocytes to remove the antigen.

As used herein, an “instructional material” includes a publication, a recording, a diagram, or any other medium of expression which can be used to communicate the usefulness of the compositions and methods of the invention. The instructional material of the kit of the invention may, for example, be affixed to a container which contains the nucleic acid, peptide, and/or composition of the invention or be shipped together with a container which contains the nucleic acid, peptide, and/or composition. Alternatively, the instructional material may be shipped separately from the container with the intention that the instructional material and the compound be used cooperatively by the recipient.

By “marker” is meant any protein or polynucleotide having an alteration in level or activity that is associated with a disease or disorder.

By the term “modified” as used herein, is meant a changed state or structure of a molecule or cell of the invention. Molecules may be modified in many ways, including chemically, structurally, and functionally. Cells may be modified through the introduction of nucleic acids.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

“Monitoring” refers to recording changes in a continuously varying parameter (e.g. monitoring progression of a disease).

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

“Parenteral” administration of an immunogenic composition includes, e.g., subcutaneous (s.c.), intravenous (i.v.), intramuscular (i.m.), or intrasternal injection, or infusion techniques.

The language “pharmaceutically acceptable carrier” includes a pharmaceutically acceptable salt, pharmaceutically acceptable material, composition or carrier, such as a liquid or solid filler, diluent, excipient, solvent or encapsulating material, involved in carrying or transporting a compound(s) of the present invention within or to the subject such that it may perform its intended function. Typically, such compounds are carried or transported from one organ, or portion of the body, to another organ, or portion of the body. Each salt or carrier must be “acceptable” in the sense of being compatible with the other ingredients of the formulation, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; diluent; granulating agent; lubricant; binder; disintegrating agent; wetting agent; emulsifier; coloring agent; release agent; coating agent; sweetening agent; flavoring agent; perfuming agent; preservative; antioxidant; plasticizer; gelling agent; thickener; hardener; setting agent; suspending agent; surfactant; humectant; carrier; stabilizer; and other non-toxic compatible substances employed in pharmaceutical formulations, or any combination thereof. As used herein, “pharmaceutically acceptable carrier” also includes any and all coatings, antibacterial and antifungal agents, and absorption delaying agents, and the like that are compatible with the activity of the compound, and are physiologically acceptable to the subject. Supplementary active compounds may also be incorporated into the compositions.

As used herein the term “podocytopenia” relates to loss, damage or degeneration of the podocytes. Podocytes are highly specialized polarized epithelial cells found in the Bowman's capsule of the kidney. Podocytes have a complex cellular morphology. The podocyte's cell body bulges into the urinary space and gives rise to long primary processes that extend toward the glomerular basement membrane (GBM) to which they attach by numerous foot processes. The foot processes of neighboring podocytes interdigitate, leaving between them filtration slits bridged by an extracellular structure, known as the slit diaphragm. In a healthy kidney, podocytes function as a filtration barrier between glomerular capillaries and the urinary space, preventing proteins from entering the urine and causing proteinuria. The slit diaphragm, which consists of a highly organized network of glycoproteins, prevents the passage of larger molecules, such as albumin, from glomerular capillaries into the urinary space. Additionally, the complex architecture of podocyte specific proteins (e.g , synaptopodin, nephrin) is required for many highly specialized functions of podocytes, which include maintenance of the capillary loop shape; counteracting the intraglomerular pressure; synthesis and maintenance of the GBM; and production and secretion of vascular endothelial growth factor (VEGF) required for glomerular endothelial cell integrity. Podocytes are the target of injury in many glomerular diseases such as such as minimal change nephropathy (MCN), membranous nephropathy (MN), focal segmental glomerulosclerosis (FSGS), collapsing glomerulopathy (CG), chronic glomerulonephritis (GN), and diabetic nephropathy (DN). Podocytopenia results in retraction of their foot processes (i.e. foot process effacement) and is associated with proteinuria (Laurens et al., Kidney Int. 1995, 47: 1078-1086); Pavenstaadt et al., Br J Pharmaco11992, 107- 189-195) and albuminuria.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

The terms “purified”, “biologically pure” or “isolated” as used herein mean having been increased in purity, wherein “purity” is a relative term, and not to be necessarily construed as absolute purity. For example, the purity of a substance, for example, but not limited to a nucleic acid, can be at least about 50%, can be greater than 60%, 70%, 80%, 90%, 95%, or can be 100%. The terms “purified”, “biologically pure” or “isolated” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

As used herein, “sample” or “biological sample” refers to anything, which may contain an analyte (e.g., polypeptide, polynucleotide, or fragment thereof) for which an analyte assay is desired. The sample may be a biological sample, such as a biological fluid or a biological tissue. In one embodiment, a biological sample is a salivary sample. Such a sample may include diverse cells, proteins, and genetic material. Examples of biological tissues also include organs, tumors, lymph nodes, arteries and individual cell(s). Examples of biological fluids include urine, blood, plasma, serum, saliva, semen, stool, sputum, cerebral spinal fluid, tears, mucus, amniotic fluid or the like.

By the term “specifically binds,” as used herein with respect to an antigen binding molecule is meant an antigen binding molecule which recognizes a specific antigen, but does not substantially recognize or bind other molecules in a sample. For example, an antigen binding molecule that specifically binds to an antigen from one species may also bind to that antigen from one or more species. But, such cross-species reactivity does not itself alter the classification of an antigen binding molecule as specific. In another example, an antigen binding molecule that specifically binds to an antigen may also bind to different allelic forms of the antigen. However, such cross reactivity does not itself alter the classification of an antigen binding molecule as specific. In some instances, the terms “specific binding” or “specifically binding,” can be used in reference to the interaction of an antigen binding molecule, an antibody, a protein, or a peptide with a second chemical species, to mean that the interaction is dependent upon the presence of a particular structure (e.g., an antigenic determinant or epitope) on the chemical species; for example, an antigen binding molecule or an antibody recognizes and binds to a specific protein structure rather than to proteins generally. If an antigen binding molecule is specific for epitope “A”, the presence of a molecule containing epitope A (or free, unlabeled A), in a reaction containing labeled “A” and the antigen binding molecule, will reduce the amount of labeled A bound to the antigen binding molecule.

The term “subject” is intended to include living organisms in which an immune response can be elicited (e.g., mammals). A “subject” or “patient,” as used therein, may be a human or non-human mammal. Non-human mammals include, for example, livestock and pets, such as ovine, bovine, porcine, canine, feline and murine mammals. Preferably, the subject is human.

A “target site” or “target sequence” refers to a genomic nucleic acid sequence that defines a portion of a nucleic acid to which a binding molecule may specifically bind under conditions sufficient for binding to occur.

The term “therapeutic” as used herein means a treatment and/or prophylaxis. A therapeutic effect is obtained by suppression, remission, or eradication of a disease state.

To “treat” a disease as the term is used herein, means to reduce the frequency or severity of at least one sign or symptom of a disease or disorder experienced by a subject. It will be appreciated that, although not precluded, treating a disorder or condition does not require that the disorder, condition or symptoms associated therewith be completely eliminated.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

The present invention relates to a novel therapeutic target, podocyte Semaphorin3a (Sema3a), for treating and preventing diabetic nephropathy in a mammal in need thereof. In one aspect, the present invention evidences that inhibiting podocyte Sema3a is critical for treating or preventing diabetic nephropathy diabetic nephropathy and its related pathologies. In another aspect, the invention provides a method for identifying or characterizing a compound useful for treating or preventing diabetes or diabetic nephropathy.

Methods of the present invention are useful for treatment of mammals, particularly humans.

In one embodiment, an agent that inhibits Sema3a is used to reduce or block Sema3a-mediated cell signaling.

As used herein the terms “Sema3a inhibiting agent”, “Sema3a inhibitor” or “Sema3a antagonist” are used interchangeably. Non-limiting examples include an agent which reduces or blocks the expression (transcription or translation) of Sema3a, an agent able to reduce or block Sema3a secretion or an agent able to reduce or block Sema3a binding to its receptor Neuropilinl (NRP1) and an agent which reduce or block transcription or translation of Npr-1. Without wishing to be limited by any theory, the agent can be natural or synthetic and can be a protein/polypeptide such as but not limited to an antibody that specifically binds to Sema3a; a soluble polypeptide or fragment thereof, a peptide, a small molecule, a polynucleotide such as but not limited to small interfering ribonucleic acids (siRNAs), micro-RNAs (mRNAs), ribozymes, and anti-sense sequences (Zeng, Proc Natl Acad Sci USA 2003, 100:9779-9784; and Kurreck , Eur J. Biochem 2003, 270:1628-1644.) specific to Sema3a nucleic acid sequence encoding a Sema3 protein.

Non limiting examples of Sema3a nucleic acid sequences are the mouse NCBI Reference Sequence: XM_006503557.2 and the human NCBI Reference Sequence: NM_006080.2. Non limiting examples of Sema3a amino acid sequences are the mouse NCBI Reference Sequence: XP_006503622.1 and the human NCBI Reference Sequence: NP_006071.1.

In one embodiment, the agent prevents Sema3a-mediated cell signaling without substantially reducing VEGF binding to the NRP1 receptor and thus VEGF- mediated cellular signaling.

In one embodiment, the inhibiting agent used in the methods of the present invention comprises a specific Sema3a inhibitor. In one embodiment, the inhibiting agent is a specific Sema3a competitive binding inhibitor. In other embodiments, the inhibitor comprises at least one from the group consisting of a natural product, a natural fungal product and a synthetic product. In yet another embodiment, wherein the inhibitor is xanthofulvin (Natural: Penicillium SPF-3059 and purified (Veron et al., J Am Soc Nephrol. 2014;25(8):1814-1824); or synthetic as described by Axelrod et al., Angew Chem Int Ed Engl. 52(12):3421-4, 2013 and in US Application No: US2014/050050).

The present disclosure demonstrates that inhibiting Sema3a reduces various pathological aspects of diabetic nephropathy not previously associated with Sema3a such as, for example, glomerular hyperfiltration. Methods for reducing or ameliorating complications associated with diabetic nephropathy pathological processes in a mammal are provided also herein.

In one embodiment, a method for treating or preventing diabetic nephropathy in a mammal in need thereof, comprises administering to the mammal a therapeutically effective amount of an agent that inhibits podocyte Sema3a. In some embodiments, the mammal has type 1 diabetes. In other embodiments, the mammal has type 2 diabetes.

In one embodiment, a method for reducing renal insufficiency in a mammal having or at risk for having diabetic nephropathy is included, where the method comprises administering to the mammal a therapeutically effective amount of an agent that inhibits podocyte Sema3a.

In another embodiment, the invention includes a method for improving kidney function in a mammal having or at risk of having impaired kidney function, where the method comprises administering to the mammal a therapeutically effective amount of an agent that inhibits podocyte Semaphorin3a (Sema3a).

Methods for assessing renal insufficiency are well established in the art. Various pathological and morphological changes can be used as markers of a diseased kidney. Such changes include but are not limited to mesangial expansion, associated with increased matrix production and accumulation of mesangial extracellular matrix; mesangial cell expansion; glomerular basement membrane thickening, which in late stage diabetic nephropathy is associated with glomerulosclerosis; and development of tubulointerstitial fibrosis. (Gilbert et al., Kidney 1999, Int 56:1627-1673). Glomerulosclerosis and tubulointerstitial fibrosis are the structural late stage kidney disease hallmarks of advanced diabetic nephropathy with renal insufficiency, resulting in reduction in glomerular filtration rate and, possibly, end stage renal disease and kidney failure.

In one embodiment, the invention includes a method for reducing podocytopenia in a mammal having or at risk for having diabetic nephropathy, comprising administering to the mammal a therapeutically effective amount of an agent that inhibits podocyte Sema3a.

In one embodiment, the invention includes a method for reducing glomerulosclerosis or glomerular filtration rate in a mammal having or at risk for having diabetic nephropathy, comprising administering to the mammal a therapeutically effective amount of an agent that inhibits podocyte Sema3a.

In another embodiment, the invention includes a method for reducing proteinuria in a mammal having or at risk for having diabetic nephropathy, comprising administering to the mammal a therapeutically effective amount of an agent that inhibits podocyte Sema3a.

In yet another embodiment, the invention includes a method for reducing albuminuria or correcting hypoalbuminemia in a mammal having or at risk for having diabetic nephropathy, comprising administering to the mammal a therapeutically effective amount of an agent that inhibits podocyte Sema3a.

As used herein, “hypoalbuminemia” is an abnormally low level of albumin in blood vessels (in general <3.5g/dL). This condition is common problem among patients with acute and chronic medical conditions.

In one embodiment, the invention includes a method of detecting inhibition of podocyte Semaphorin3a (Sema3a). The method comprises measuring the level of gene expression in the kidney of at least one biomarker selected from the group consisting of, laminin, creatinine, albumin, collagen IV, metalloproteinases 2 and 9, VEGF-A, VEGFR2, αvβ3 integrin, PlexinAl and MICAL in a mammal treated with an inhibitor of Sema3a and in an otherwise identical mammal not treated with the inhibitor. When the level of the at least one biomarkers is increased or decreased in the treated mammal as compared with the untreated mammal, Sema3a is inhibited.

In one embodiment, the invention includes a method of detecting inhibition of podocyte Semaphorin3a (Sema3a). The method comprises measuring the plasma and/or urine level of at least one polypeptide selected from the group consisting of creatinine and albumin in a mammal treated with an inhibitor of Sema3a and in an otherwise identical mammal not treated with the inhibitor. When the level of the at least one polypeptide is decreased in the treated mammal as compared with the untreated mammal, Sema3a is inhibited.

In some embodiments, the level of the biomarkers between the treated mammal and the otherwise identical mammal not treated is increased or decreased by at least 1.1 fold. In other embodiments, this increase or decrease is by at least 10 fold. In other embodiments, the mammal has diabetic nephropathy. In yet other embodiments, the mammal has a type 1 and/or a type 2 diabetes.

### Combination Therapies

The compounds identified in the methods described herein may also be useful in the methods of the invention when combined with at least one additional compound useful for treating diabetes and/or diabetic neuphrotathy. The additional compound may comprise a compound identified herein or a compound, e.g., a commercially available compounds, known to treat, prevent, or reduce the symptoms of diabetes and/or diabetic neuphrotathy.

In one aspect, the present invention contemplates that the agents useful within the invention may be used in combination with a therapeutic agent such as an anti-diabetic agent.

In some embodiment, the methods of the present invention comprise further administering to the mammal an additional hypoglycemic or any anti-diabetic therapeutic agents known in the art. Examples of additional anti-diabetic therapeutic agents include, but are not limited to, a-glucosidase inhibitors, lipase inhibitors, sulfonyl ureas, meglitinides, biguanides, thiazolidinediones, pramlintide, incretin mimetics, GLP-1 receptor agonists, DPP-IV inhibitors, asprin, niacin, fibrates, bile acid sequestrants, cholesterol absorption inhibitors, omega-3 acid ethyl esters, secretory phospholipase A2 (“sPLA2”) inhibitors, oligonucleotide-based apolipoprotein B (“apoB”) inhibitors, squalene synthase inhibitors, statins, fixed dose combination statin therapies, glucose, glucagon, heparin, angiotensin II receptor antagonists, ACE inhibitors, antidepressants, anticonvulsants, opioids and opioid-like drugs, C-peptide, aldose reductase inhibitors, pancreatic lipase inhibitors, Serotonin-norepinephrine reuptake inhibitors, and cannabinoid (“CB1”) receptor antagonists, leptin receptor agonists, oxyntomodulin or an oxyntomodulin-derived peptide, peptide tyrosine-tyrosine (PYY), anti-obesity therapies, anti-obesity combination therapies, erectile dysfunction medications, alpha-1-adrenergic receptor blockers, 5-alpha reductase inhibitors, fish oil, plant sterols and stanols, immunosuppressors, SGLT2 inhibitors, 11βHSD1 inhibitors, adenosine Al receptor agonists, anti-inflammatory agents, artificial sweeteners, bile acid receptor agonists, CCK receptor antagonists, CCR2 antagonists, diacylglycerol O-acyltransferase homolog 1 (DGAT-1) inhibitors, dopamine receptor agonists, dual-acting peptide-GLP-1 and glucagons receptor agonists, FGF-21 variants, fructose 1,6 bisphosphatase inhibitors, gastrin-releasing peptide (GRP) receptor agonists, GLP-1 analogs, glucagons receptor-antisense, glucokinase activators, glucose-dependent insulinotropic receptor (GDIR/GPR119) agonists, glutamic acid decarboxylases, HM74a agonists, HSP60 peptides, IL-1 antibody (Eli Lilly), insulin-derived peptides, longer acting human GLP-1 analogues, Monoclonal antibodies (“MAbs”) to CD3, MAbs to glucagon receptors, MAbs to IL-1, MAbs to IL-1β, permeability inhibitors, plasmid encoding proinsulins, poly(ADP-ribose) polymerase inhibitors, PPAR agonists, PPAR alpha activators, PPAR gamma modulators, PPAR pan agonists, PPARα/γ modulators, protein tyrosine phosphatase 1B inhibitor, SGLT1 inhibitors, SIAC (soluble insulin analogue combination, soluble insulin basal analogues, sirtuin (SIRT1) activators, sodium channel blockers, and other non-insulin compounds.

### Pharmaceutical Compositions and Formulations.

The invention includes the use of a pharmaceutical composition comprising a Sema3a inhiting agent as described herein for use in the methods of the invention.

Such a pharmaceutical composition is in a form suitable for administration to a subject, or the pharmaceutical composition may further comprise one or more pharmaceutically acceptable carriers, one or more additional ingredients, or some combination of these. The various components of the pharmaceutical composition may be present in the form of a physiologically acceptable salt, such as in combination with a physiologically acceptable cation or anion, as is well known in the art.

In an embodiment, the pharmaceutical compositions useful for practicing the method of the invention may be administered to deliver a dose of between 1 ng/kg/day and 100 mg/kg/day. In another embodiment, the pharmaceutical compositions useful for practicing the invention may be administered to deliver a dose of between 1 ng/kg/day and 500 mg/kg/day.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

Pharmaceutical compositions that are useful in the methods of the invention may be suitably developed for inhalational, oral, rectal, vaginal, parenteral, topical, transdermal, pulmonary, intranasal, buccal, ophthalmic, intrathecal, intravenous or another route of administration. Other contemplated formulations include projected nanoparticles, liposomal preparations, resealed erythrocytes containing the active ingredient, and immunologically-based formulations. The route(s) of administration is readily apparent to the skilled artisan and depends upon any number of factors including the type and severity of the disease being treated, the type and age of the veterinary or human patient being treated, and the like.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

As used herein, a “unit dose” is a discrete amount of the pharmaceutical composition comprising a predetermined amount of the active ingredient. The amount of the active ingredient is generally equal to the dosage of the active ingredient that would be administered to a subject or a convenient fraction of such a dosage such as, for example, one-half or one-third of such a dosage. The unit dosage form may be for a single daily dose or one of multiple daily doses (e.g., about 1 to 4 or more times per day). When multiple daily doses are used, the unit dosage form may be the same or different for each dose.

Although the descriptions of pharmaceutical compositions provided herein are principally directed to pharmaceutical compositions suitable for ethical administration to humans, it is understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, and dogs.

In one embodiment, the compositions are formulated using one or more pharmaceutically acceptable excipients or carriers. In one embodiment, the pharmaceutical compositions comprise a therapeutically effective amount of Sema3a inhiting agent and a pharmaceutically acceptable carrier. Pharmaceutically acceptable carriers, which are useful, include, but are not limited to, glycerol, water, saline, ethanol and other pharmaceutically acceptable salt solutions such as phosphates and salts of organic acids. Examples of these and other pharmaceutically acceptable carriers are described in Remington's Pharmaceutical Sciences, 1991, Mack Publication Co., New Jersey.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

The composition of the invention may comprise a preservative from about 0.005% to 2.0% by total weight of the composition. The preservative is used to prevent spoilage in the case of exposure to contaminants in the environment. Examples of preservatives useful in accordance with the invention included but are not limited to those selected from the group consisting of benzyl alcohol, sorbic acid, parabens, imidurea and combinations thereof. A particularly preferred preservative is a combination of about 0.5% to 2.0% benzyl alcohol and 0.05% to 0.5% sorbic acid.

The composition preferably includes an antioxidant and a chelating agent which inhibit the degradation of the compound. Preferred antioxidants for some compounds are BHT, BHA, alpha-tocopherol and ascorbic acid in the preferred range of about 0.01% to 0.3% and more preferably BHT in the range of 0.03% to 0.1% by weight by total weight of the composition. Preferably, the chelating agent is present in an amount of from 0.01% to 0.5% by weight by total weight of the composition. Particularly preferred chelating agents include edetate salts (e.g. disodium edetate) and citric acid in the weight range of about 0.01% to 0.20% and more preferably in the range of 0.02% to 0.10% by weight by total weight of the composition. The chelating agent is useful for chelating metal ions in the composition which may be detrimental to the shelf life of the formulation. While BHT and disodium edetate are the particularly preferred antioxidant and chelating agent respectively for some compounds, other suitable and equivalent antioxidants and chelating agents may be substituted therefore as would be known to those skilled in the art.

### Administration/Dosing

The regimen of administration may affect what constitutes an effective amount. For example, the therapeutic formulations may be administered to the patient either prior to or after a surgical intervention related to diabetes or diabetic nephropathy, or shortly after the patient was diagnosed with a diabetes or diabetic nephropathy. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a patient, preferably a mammal, more preferably a human, may be carried out using known procedures, at dosages and for periods of time effective to treat diabetic nephropathy in the patient. An effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the patient being treated, and like factors well-known in the medical arts. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the invention is from about 0.01 and 50 mg/kg of body weight/per day. One of ordinary skill in the art would be able to study the relevant factors and make the determination regarding the effective amount of the therapeutic compound without undue experimentation.

The compound can be administered to an animal as frequently as several times daily, or it may be administered less frequently, such as once a day, once a week, once every two weeks, once a month, or even less frequently, such as once every several months or even once a year or less. It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on. The frequency of the dose is readily apparent to the skilled artisan and depends upon any number of factors, such as, but not limited to, the type and severity of the disease being treated, and the type and age of the animal. Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient. A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of diabetic nephropathy in a patient.

### Routes of Administration

One skilled in the art will recognize that although more than one route can be used for administration, a particular route can provide a more immediate and more effective reaction than another route.

Routes of administration of any of the compositions of the invention include inhalational, oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, subcutaneous, intramuscular, intradermal, intra-arterial, intravenous, intrabronchial, inhalation, and topical administration. Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

In one embodiment, the administration route is a continuous subcutaneous administration for at least 2 days. In another embodiment, the administration route is a continuous subcutaneous administration for at least 20 days. In yet another embodiment, the administration route is a continuous subcutaneous administration for at least 30 days.

### Controlled Release Formulations and Drug Delivery Systems

Controlled- or sustained-release formulations of a pharmaceutical composition of the invention may be made using conventional technology. In some cases, the dosage forms to be used can be provided as slow or controlled-release of one or more active ingredients therein using, for example, hydropropylmethyl cellulose, other polymer matrices, gels, permeable membranes, osmotic systems, multilayer coatings, microparticles, liposomes, or microspheres or a combination thereof to provide the desired release profile in varying proportions. Suitable controlled-release formulations known to those of ordinary skill in the art, including those described herein, can be readily selected for use with the pharmaceutical compositions of the invention. Thus, single unit dosage forms suitable for oral administration, such as tablets, capsules, gelcaps, and caplets, which are adapted for controlled-release are encompassed by the present invention.

Most controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood level of the drug, and thus can affect the occurrence of side effects.

Screening assays for a Sema3a Inhibitor

The invention further includes the use of Sema3a as a target in screening assays used to identify a compound potentially useful for treating or preventing diabetic nephropathy.

In one embodiment, the present invention provides a method for identifying or characterizing a compound useful for treating or preventing diabetic nephropathy in a mammal in need thereof. The method comprises measuring the level of expression or activity of at least one of podocyte Semaphorin3a (Sema3a) nucleic acid, Sema3a encoded polypeptide, Sema3a receptor and any combination thereof, in a mammal treated with the compound and in an otherwise identical mammal not treated with the compound. When the level of expression or activity of at least one of podocyte Sema3a nucleic acid, Sema3a encoded polypeptide, Sema3a receptor and any combination thereof, is decreased in the treated mammal as compared to the untreated mammal, the compound is useful for treating or preventing diabetic nephropathy.

The identification of any such compound may be accomplished using several methods known in in art. In one non-limiting example, a reporter assay-based method of selecting agents which modulate Sema3a expression can be used. This method includes providing a cell comprising a nucleic acid sequence comprising a Sema3a transcriptional regulatory sequence operably-linked to a suitable reporter gene. The cell is then exposed to the agent suspected of affecting Sema3a expression (e.g., a test/candidate compound) and the transcription efficiency is measured by the activity of the reporter gene. The activity can then be compared to the activity of the reporter gene in cells unexposed to the agent in question. Suitable reporter genes include but are not limited to beta-galactosidase, luciferase, chloramphenicol acetyltransferase and green fluorescent protein (GFP). Such assay systems may comprise a variety of means to enable and optimize useful assay conditions. Such means may include but are not limited to: suitable buffer solutions, for example, for the control of pH and ionic strength and to provide any necessary components for optimal Sema3a activity and stability (e.g., protease inhibitors), temperature control means for optimal Sema3a activity and or stability, and detection means to enable the detection of the Sema3a and NRP1 interaction. A variety of such detection means may be used, including but not limited to one or a combination of the following: radiolabelling (e.g., ³²P, ¹⁴C, ³H), antibody-based detection, fluorescence, chemiluminescence, spectroscopic methods (e.g., generation of a product with altered spectroscopic properties), various reporter enzymes or proteins (e.g., horseradish peroxidase, green fluorescent protein), specific binding reagents (e.g., biotin/streptavidin), and others. The assay may be carried out in vitro utilizing a source of Sema3a which may comprise naturally isolated or recombinantly produced Sema3a, in preparations ranging from crude to pure. Recombinant Sema3a may be produced in a number of prokaryotic or eukaryotic expression systems, which are well known in the art (Martin F. et al., 2001. Immunogenetics 53(4): 296-306) for the recombinant expression of Sema3a. Such assays may be also performed in an large scale array format.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

The materials and methods employed in these experiments are now described.

### Human Kidney Biopsy Studies

De-identified human kidney biopsy frozen sections diagnosed with class III or IV diabetic nephropathy (n=6, 1 T1D, 2 T2D, 3 unspecified DM) or non-diabetic renal disease (n=4, 1 hypertension, 1 obesity, 2 proteinuria) were obtained from NephroCorTM following IRB approval of the study. Sema3a and podocin fluorescent immunohistochemistry were performed as described (Veron et al., Diabetologia 2011;54(5):1227-1241).

### Animal Studies

Podocin-rtTA:tet-O-Sema3a mice, herein called Sema3a⁺, generated previously (Reidy et al., Development 2009;136(23):3979-3989) were maintained in FVB genetic background for >10 generations. Diabetes was induced (low dose protocol, AMDCC.org) in 6-8 weeks old male Sema3a⁺ mice by 5 daily i.p. streptozotocin (STZ) (50 mg/kg) . Diabetes (random blood glucose >300 mg/dl) was confirmed using glucose oxidase biosensor blood glucose meter (One-Touch-Ultra-2; LifeScan) one week after the last STZ injection. Diabetic Sema3a⁺ (DM-Sema3a⁺) (n=15) and non-diabetic Sema3a⁺ (non-DM-Sema3a⁺) (n=18) mice were fed doxycycline-containing diet (0.625mg/kg chow, Harlan-Teklad) or standard chow for 12-16 weeks. After 12-16 weeks of diabetes, 24 h urine was collected in metabolic cages, blood and kidneys were collected under anesthesia. For Sema3a inhibition studies, DM-Sema3a⁺ mice were fed doxycycline-containing chow for 8 weeks, then Alzet osmotic pumps (Model 1004) containing xanthofulvin (0.5 mg/ml in PBS) (Ieda et al., Nat Med 2007;13(5):604-612) (n=4) or saline (n=2) were implanted subcutaneously, all mice were kept on doxycycline-containing chow for the following 4 weeks.

### Generation of Conditional Podocyte-Specific PlexinA1 Knockout Mice

To delete plexinA1 selectively in podocytes in a doxycycline dependent manner, previously reported plexinA1^{+/fl} mice (Perl et al., Proc Natl Acad Sci USA 2002; 99(16): 10482-10487) were bred with tet-Cre mice (Takegahara et al., Nat Cell Biol. 2006;8(6):615-22) and double heterozygous were bred to Sema3a⁺ mice and maintained on FVB background. Quadruple transgenic mice (plexinA1^{fl/fl}:tet-O-Cre:podocin-rtTA:tet-O-Sema3a) (n=6) and their double or triple transgenic littermates lacking the tet-regulated transgenes were made diabetic (n=5), fed doxycycline containing diet and examined after 12 weeks, following the protocol described above. Genotyping was performed by PCR using previously reported primers (Reidy et al., Development 2009;136(23):3979-3989; Perl et al., Proc Natl Acad Sci USA 2002; 99(16): 10482-10487; Takegahara et al., Nat Cell Biol. 2006;8(6):615-22). All animal protocols were approved by the Yale Animal Care and Use Committee.

### Renal Phenotype Analysis

Urinary albumin was measured using mouse albumin ELISA (Bethyl Laboratories) and SDS-PAGE/Coomassie Blue staining as described (Reidy et al., Am J Pathol 2013;183(4):1156-1168). Plasma and 24h urine creatinine were measured by HPLC. Transmission electron microscopy (TEM) was performed using standard techniques. Glomerular area was measured using ImageJ-NIH (rsb.info.nih.gov/ij/) in 34±2.1 glomeruli/kidney from 4 mice/experimental condition. A renal pathologist (G.M.) examined PAS stained kidney specimens in blinded fashion and assigned a semiquantitative pathology score based on % area (0=none;1=1-25%;2=26-50%;3=51-75%;4=76-100%) with the following features: glomerular nodules, mesangiolysis, mesangial sclerosis and interstitial fibrosis. The percentage glomeruli/section containing mesangiolysis or nodules was calculated (Veron et al., J Am Soc Nephrol. 2014;25(8):1814-1824).

### Mouse Plasma and Urine Sema3a ELISA

Plasma and urine samples appropriately diluted were dispensed into microtiter plates and incubated overnight at 4° C. Plates were washed, blocked with 5% powdered milk in wash buffer, incubated with Sema3a antibody (sc-1148, Santa Cruz Biotechnology) for 2 h at 37° C., followed by extensive washes, peroxidase-conjugated rabbit anti-goat IgG (305-035-003, Jackson ImmunoResearch) for 1 h at 37° C., washes followed by peroxidase substrate (34022, Pierce) for 30 min, 2M H₂SO₄. O.D. was measured at 450 nm using a microplate reader (BioRad). Recombinant mouse Sema3a (Reidy et al., Development 2009;136(23):3979-3989) served as standard.

### Immunohistochemistry

Fluorescent immunostaining studies were performed on frozen kidney sections, as described (Reidy et al., Development 2009;136(23):3979-3989) using the following primary antibodies: anti-laminin (L9393; Sigma), collagenIV (Southern Biotech), Sema3a (R&D AF1250), nephrin (20R-NP002; Fitzgerald Inc.), WT1 (sc-192; Santa Cruz), podocin (P0372; Sigma) and αvβ3 integrin (EMD-Millipore). Dual-immunolabeling was performed using appropriate Cy2 and Cy3 fluorescent-tagged secondary antibodies (Jackson ImmunoResearch Laboratories), signals were visualized by confocal microscopy (FluoView 300; Olympus), and quantitated using ImageJ (Reidy et al., Am J Pathol 2013;183(4):1156-1168).

### Immunoblot Analysis

Equal amount of protein from ≧4 kidney lysates/experimental condition were pooled and resolved by SDS-PAGE, immunoblotting was performed using standard technique with the following primary antibodies: WT1, podocin, nephrin, laminin, Sema3a (sc-28867; Santa Cruz), β3-integrin (sc-14009; Santa Cruz), β1-inte_{g}rin (AB1952; EMD-Millipore), neuropilin-1 (Kolodkin et al., Cell 1997;90:753-762; Takahashi et al., Cell 1999;99(1):59-69), MMP-2 (MAB13434; EMD-Millipore), MMP-9 (AB19016; EMD-Millipore), plexinA₁ (sc-25639; Santa Cruz), VEGFR-2 (2479; Cell Signaling), MICAL1 (14818-1-AP; Proteintech); actin (A2066; Sigma) or tubulin (Sigma) were used as loading controls. Signals were detected with appropriate HRP-conjugated secondary antibodies, visualized by chemiluminescence and quantified using ImageJ.

### PlexinA₁-MICAL1 Co-Immunoprecipitation

Sema3a⁺ podocytes were lysed in immuno-precipitation buffer as described (Reidy et al., Am J Pathol 2013;183(4):1156-1168). Lysates were pre-cleared, incubated with MICAL1 antibody (14818-1-AP; Proteintech), incubated overnight with protein A-agarose, eluted, analyzed by immunoblotting using PlexinA₁, MICAL1 and actin antibodies, detected by ECL. Rabbit serum served as negative control, lysate from MICAL1/plexinA₁ transfected HEK cells served as positive control.

### Podocyte Morphology Assay

Sema3a⁺ podocyte cell line was described previously (Reidy et al., Am J Pathol 2013;183(4):1156-1168). Sema3a⁺ podocytes were exposed to RPMI 1640 medium+1%FBS, medium+0.1 μM xanthofulvin, medium+100 ng/ml rat recombinant Sema3a (Reidy et al., Development 2009;136(23):3979-3989) or medium+xanthofulvin+Sema3a for 16 h, fixed and stained with rhodamine-phalloidin. Images were acquired (Zeiss Axiovert); podocyte area (μm²) was measured using Zeiss AxioVision software as described (Reidy et al., Am J Pathol 2013;183(4):1156-1168), n=71±4 cells/experimental condition from 3 independent experiments were measured.

### Podocyte MICAL1 Knockdown and Morphology Assay.

Podocyte MICAL-1 knockdown was induced using a mouse MICAL-1 siRNA oligonucleotide (CAGGUGCCAUGACUAAGUAUU, SEQ ID NO: 1) (Zhou et al., Mol Cell Biol 2011;31(17):3603-3615) (Dharmacon). Briefly, podocytes were transfected with 200 pmol MICAL-1 siRNA or scrambled siRNA using Oligofectamine (Invitrogen) and incubated for 72 hours. MICAL1 knockdown was confirmed by immunoblot. Podocytes with or without MICAL1 knockdown were exposed to mouse recombinant Sema3a (100 ng/ml) for 6 hours. Podocytes were fixed, permeabilized, stained with rhodamine-phalloidin, and their morphology was analyzed as described above.

### Statistical Analysis.

Data are expressed as mean±SEM. Student's unpaired t-test or ANOVA were used to compare experimental groups, as appropriate. Linear association between two variables was evaluated by Pearson correlation and association between categorical variables was assessed by Fischer exact test. P<0.05 was deemed statistically significant.

The results of the experiments are now described in the following examples.

### Example 1: Podocyte SEMA3A is Increased in Human Diabetic Nephropathy

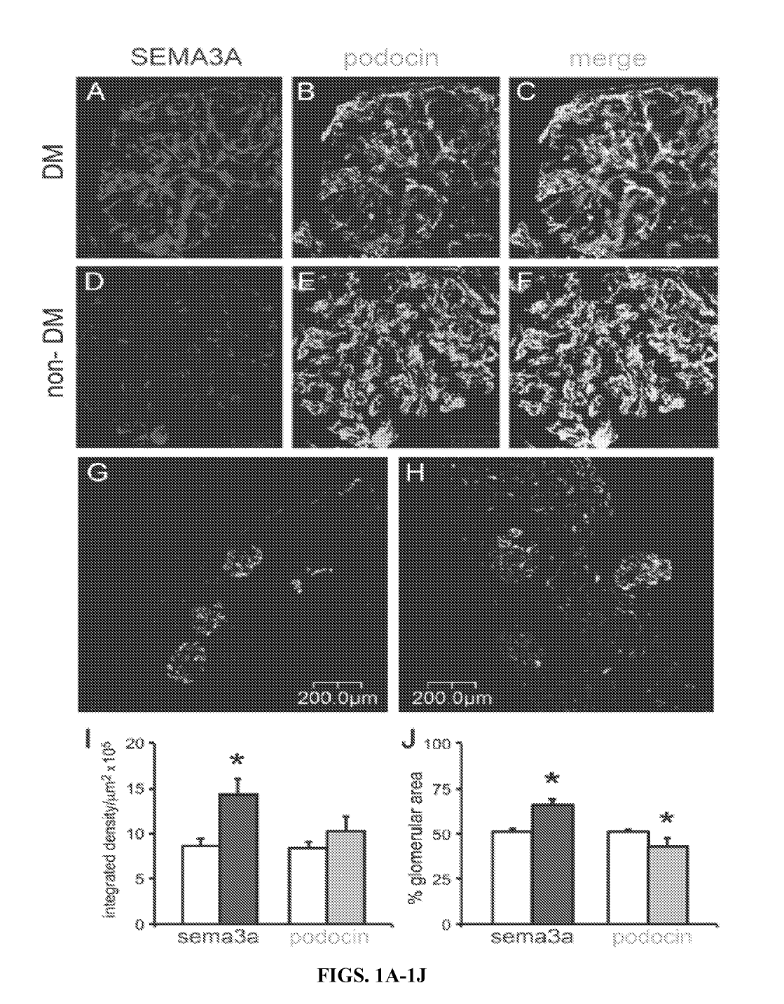

Dual-immunostaining of renal biopsy sections from type 1 and type 2 diabetes patients with Class III and Class IV diabetic nephropathy (n=6) with Sema3a and podocin antibodies revealed significantly more immunoreactive SEMA3A localized to podocytes (FIGS. 1A-1C and FIG. 1H) than in non-diabetic patients (n=4) with hypertension, obesity or proteinuria due to minimal change disease (FIGS. 1D-1G). Immunoreactive SEMA3A was also detected in renal tubules in all biopsies examined but was not differentially expressed in diabetic and non-diabetic specimens. Quantitation of immunofluorescent signals showed almost two-fold higher glomerular SEMA3A in diabetic than in non-diabetic glomeruli (FIG. 1I and FIG. 1J).

### Example 2: Sema3a⁺ Gain-of-Function Increases Plasma and Urine Sema3a in Diabetic Mice

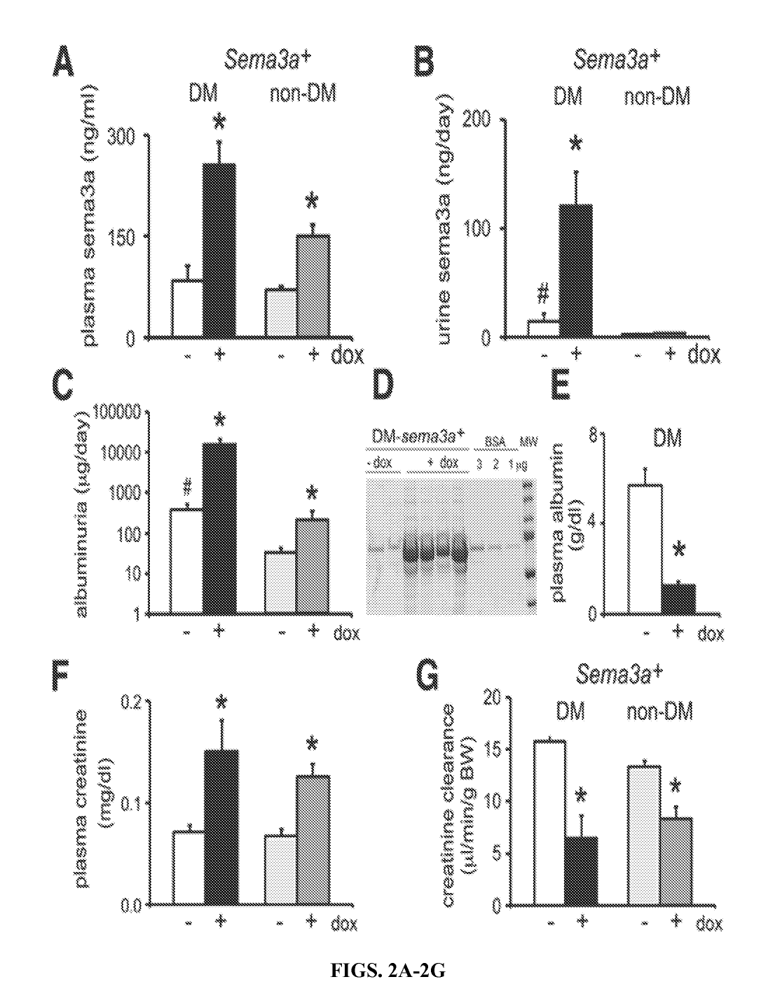

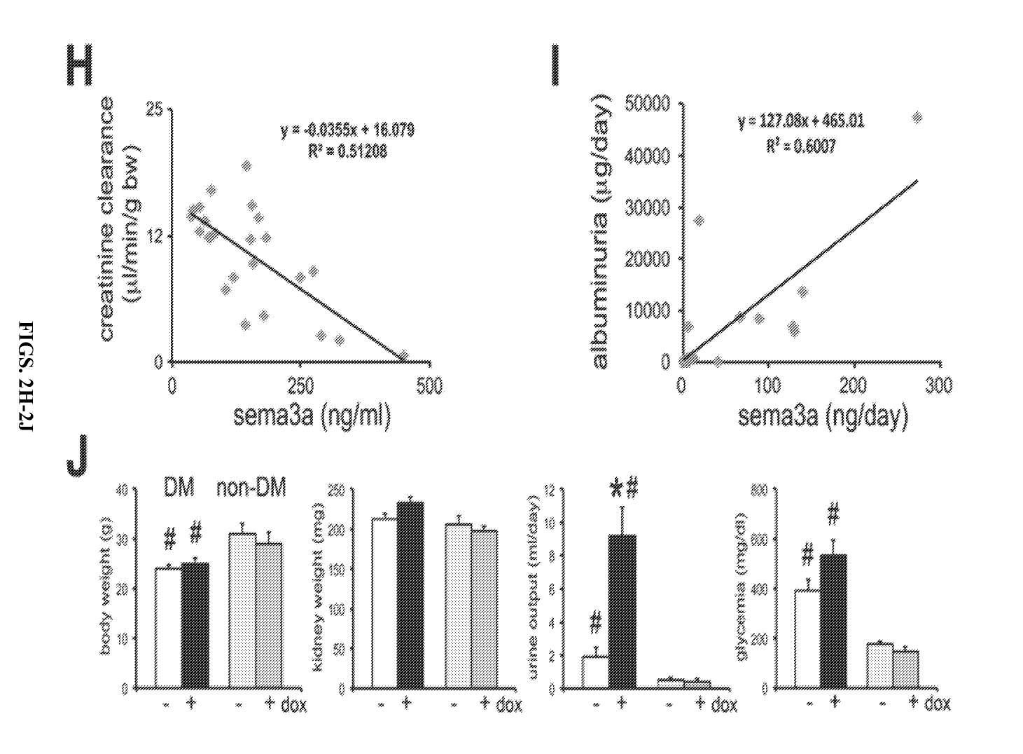

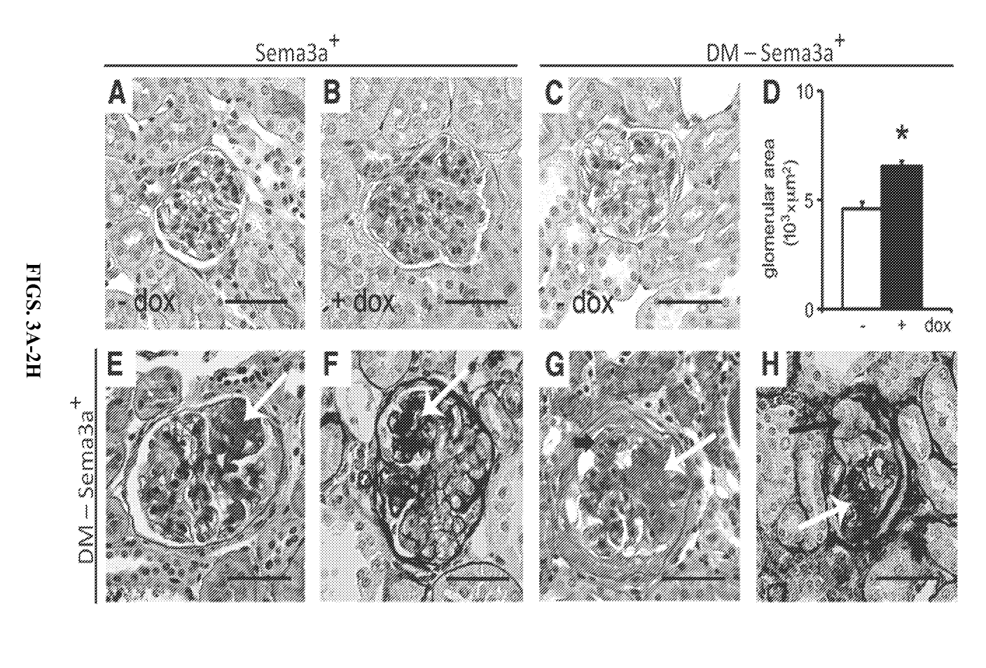

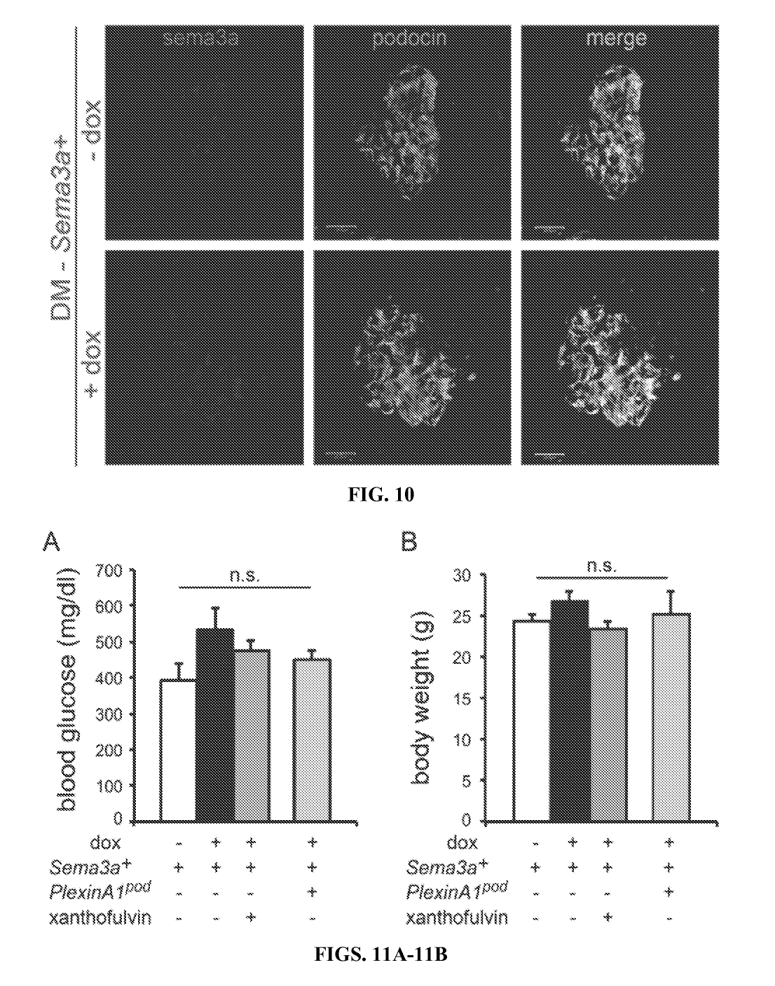

To determine whether excess podocyte Sema3a influences the severity of diabetic nephropathy, an inducible podocyte Sema3a⁺ gain-of-function mouse model that increases renal Sema3a 2 to 4-fold (Reidy et al., Development 2009;136(23):3979-3989) was used and made diabetic by low dose streptozotocin (AMDCC protocol) (Veron et al., Diabetologia 2011;54(5):1227-1241). Diabetic and non-diabetic genetically identical mice were fed doxycycline-containing or standard chow for 12-16 weeks. Plasma Sema3a level was similar in control diabetic and non-diabetic mice (FIG. 2A), suggesting that the diabetic milieu, per se, does not increase Sema3a. In contrast, podocyte Sema3a⁺ gain-of-function increased Sema3a plasma level in both non-diabetic and diabetic mice (FIG. 2A), suggesting that podocyte Sema3a secretion is a significant determinant of Sema3a plasma levels. Sema3a increase was larger (˜Three fold vs. two fold) in diabetic mice than in non-diabetic mice, likely due to changes in clearance. Podocyte Sema3a⁺ gain-of-function increased Sema3a excretion ˜8-fold and urine output increased ˜4 fold in diabetic mice, whereas no Sema3a excretion change was detected in non-diabetic mice (FIG. 2B and FIG. 2J), suggesting that diabetes exacerbates Sema3a secretion. Glomerular immunoreactive Sema3a was increased in Sema3a⁺ diabetic mice (FIG. 10).

### Example 3: Sema3a⁺ Gain-of-Function in Diabetic Mice Causes Massive Proteinuria and Renal Failure

Induction of podocyte Sema3a⁺ gain-of-function in diabetic mice dramatically exacerbates albuminuria ˜40-fold higher than diabetic control (FIGS. 2C-2D, note logarithmic scale in FIG. 2C). This massive proteinuria results in nephrotic syndrome, indicated by associated hypoalbuminemia (FIG. 2E). In contrast, non-diabetic Sema3a⁺ gain-of-function mice develop modest albuminuria (FIG. 2C), and their plasma albumin remains normal (4.8±0.65 g/dl). Podocyte Sema3a⁺ gain-of-function in diabetic mice decreases creatinine clearance by ˜60% and increases plasma creatinine >two-fold (FIGS. 2F-2G), while creatinine clearance decreases ˜35% in non-diabetic mice. Creatinine clearance correlates inversely with plasma Sema3a (FIG. 2H) and albuminuria correlates directly with Sema3a urine excretion (FIG. 2I). General parameters are shown in FIG. 2J.

### Example 4: Sema3a⁺ Gain-of-Function in Diabetic Mice Causes Advanced Diabetic Nephropathy

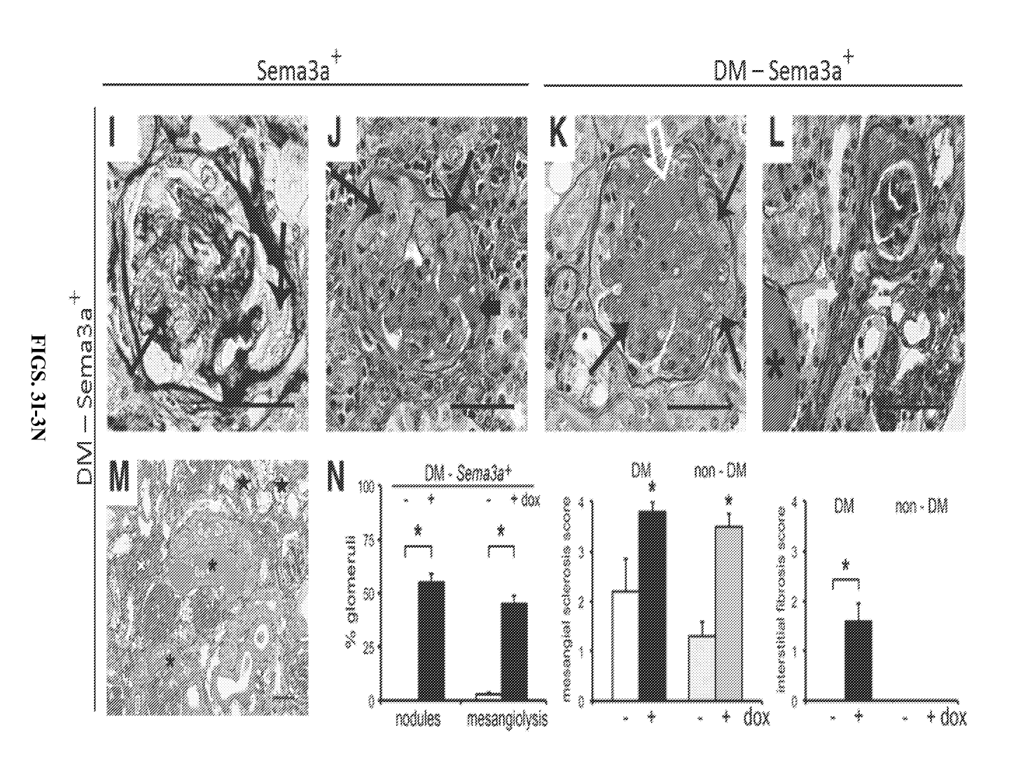

Examination of uninduced non-diabetic kidneys showed normal histology (FIG. 3A), while Sema3a⁺ gain-of-function induction resulted in mesangial expansion (FIG. 3B), as previously described (16). Uninduced diabetic kidneys (DM-Sema3a⁺−Ldox) showed mesangial expansion and glomerulomegaly, the expected mild STZ-induced diabetic nephropathy phenotype (FIGS. 3C-3D). In contrast, diabetic Sema3a⁺ gain-of-function kidneys (DM-Sema3a⁺+dox) with identical genotype revealed extensive mesangiolysis, nodular and diffuse glomerulosclerosis, as well as mesangial sclerosis, arteriolar hyalinosis, interstitial fibrosis, protein casts and fibrin caps, consistent with advanced diabetic nephropathy (FIGS. 3E-3M). Blinded morphometric analysis confirmed these observations, summarized in a semi-quantitative pathological score including glomerular nodules, mesangiolysis, mesangial sclerosis and interstitial fibrosis (FIG. 3N). Quantitation of Kimmelstiel-Wilson-like nodules showed that 55±4% of glomeruli from diabetic Sema3a⁺ gain-of-function mice have PAS-positive nodules, whereas no nodules were observed in glomeruli from control diabetic mice (0±0%) (FIG. 3N). The non-random association of Sema3a⁺ gain-of-function with glomerular nodules and mesangiolysis in diabetic mice was confirmed by Fischer's exact tests (p=0.0079 and p=0.047, respectively). Together, these findings demonstrate that Sema3a⁺ gain-of-function induces diabetic nodular glomerulosclerosis and advanced diabetic nephropathy.

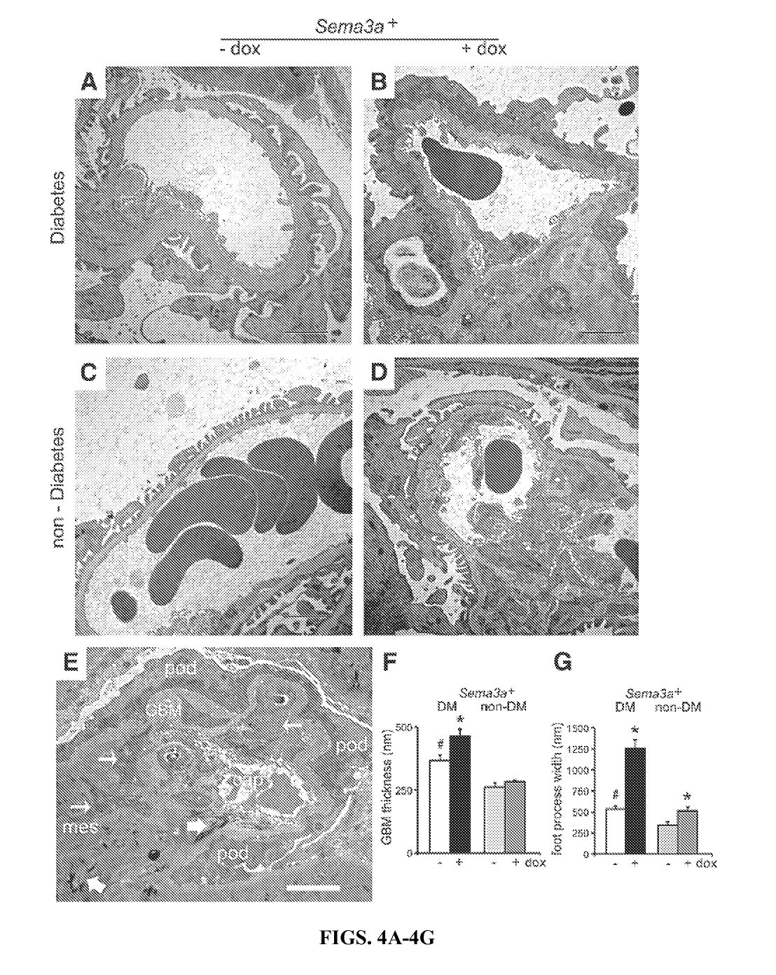

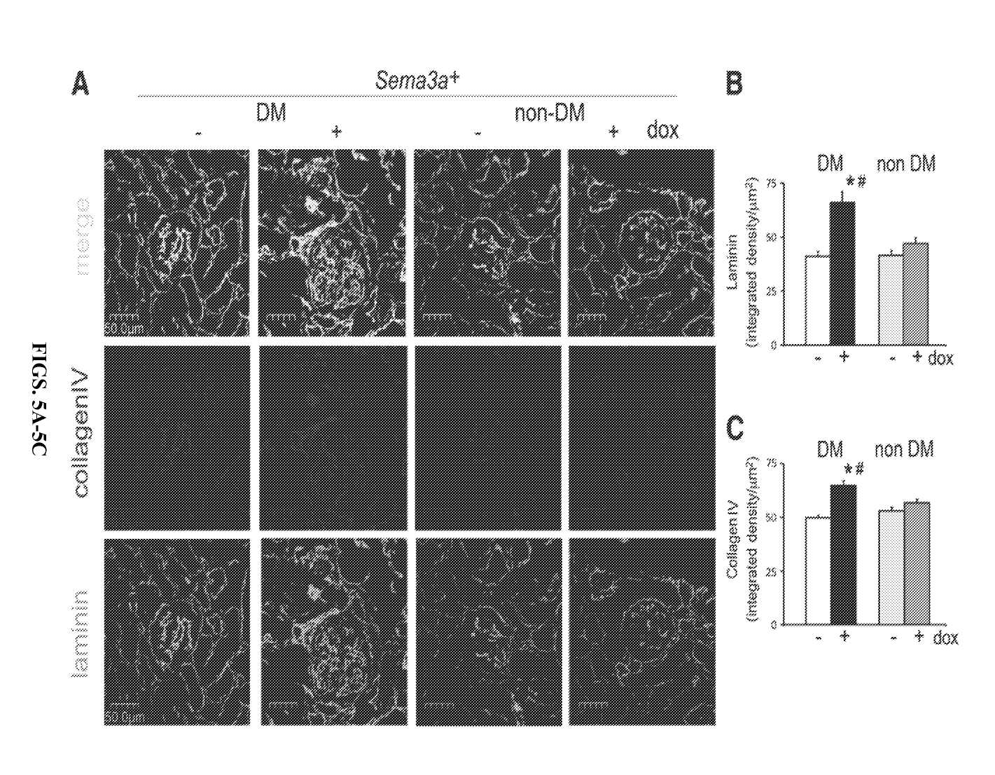

Transmission electron microscopy of diabetic control kidneys showed mild, focal foot process effacement and GBM thickening (FIG. 4A). In contrast, Sema3a⁺ gain-of-function diabetic kidneys revealed diffuse foot process effacement, podocyte vacuolae, absence of slit-diaphragms, GBM thickening (FIG. 4B) and endothelial injury consisting of glomerular endothelial cell swelling, detachment with expansion of subendothelial space and capillary lumen narrowing, as well as extensive mesangial sclerosis and fibrillar electron dense deposits in some nodules (FIG. 4E). No microthrombi or fibrin deposition was observed. Uninduced non-diabetic Sema3a⁺ mice showed normal glomerular ultrastructure (FIG. 4C). In non-diabetic mice Sema3a⁺ gain-of-function induced significantly less severe podocyte and endothelial cell lesions (FIG. 4D). Ultrastructural abnormalities quantitation showed that all diabetic kidneys had significantly thicker GBM than non-diabetic ones, Sema3a⁺ gain-of-function caused further GBM thickening and >3-fold increase of podocyte foot process width in diabetic mice, as compared with diabetic and non-diabetic controls (FIG. 4F and FIG. 4G). Collectively, TEM findings indicate that Sema3a⁺ gain-of-function in diabetic mice exacerbates the hallmark features of diabetic nephropathy, leading to class IV-like diabetic nodular glomerulosclerosis.

### Example 5: Laminin and Collagen IV are Increased in DM-Sema3a⁺ Glomerular Nodules

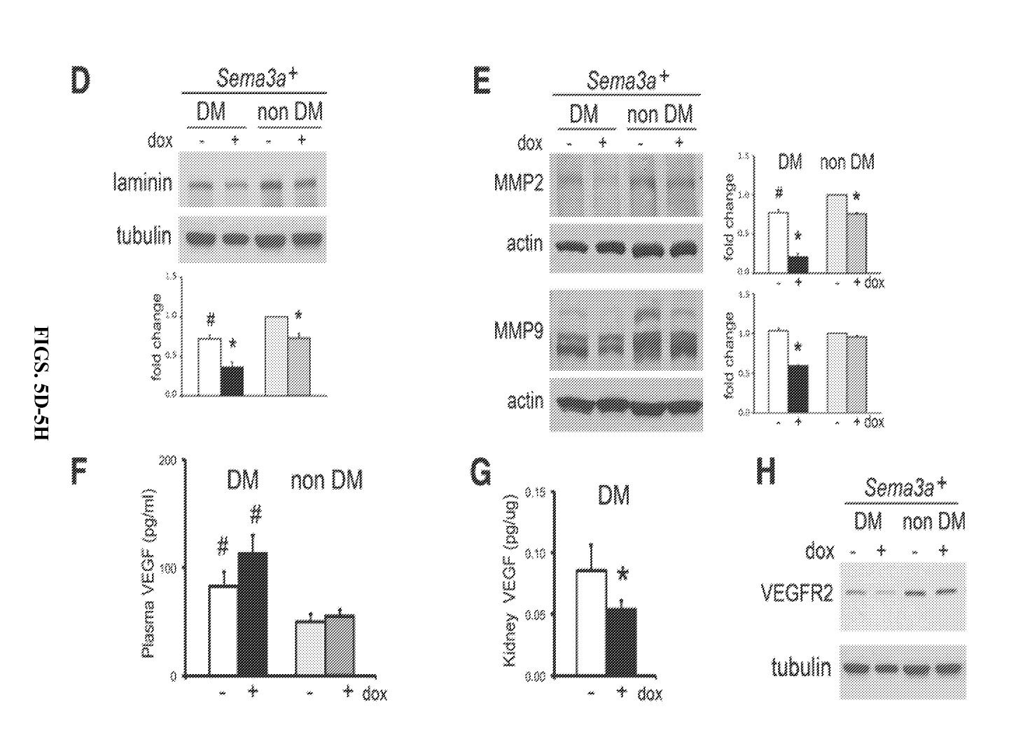

Laminin and collagen IV were significantly increased in Sema3a⁺ gain-of-function diabetic mice glomeruli (FIG. 5A-C), although total kidney laminin was decreased (FIG. 5D). Expression of metalloproteinases 2 and 9, major glomerular collagenases, was downregulated in Sema3a⁺ gain-of-function (FIG. 5E), consistent with the collagen IV accumulation observed by immunohistochemistry. Since podocyte VEGF-A gain-of-function in diabetic mice causes nodular glomerulosclerosis (Veron et al., Diabetologia 2011;54(5):1227-1241), VEGF-A expression in Sema3a⁺ diabetic mice was examined. Plasma VEGF-A was higher in all diabetic mice (irrespective of transgene induction) than in non-diabetic mice, but kidney VEGF-A and VEGFR2 expression were downregulated in Sema3a⁺ gain-of-function diabetic mice (FIGS. 5F-5H), indicating that excess VEGF-A is not a determinant of glomerular nodule development in diabetic Sema3a⁺ gain-of-function mice.

### Example 6: Sema3a⁺ Gain-of-Function Downregulates Nephrin, WT-1 and αvβ3 Integrin and Accentuates Podocytopenia in Diabetic Mice

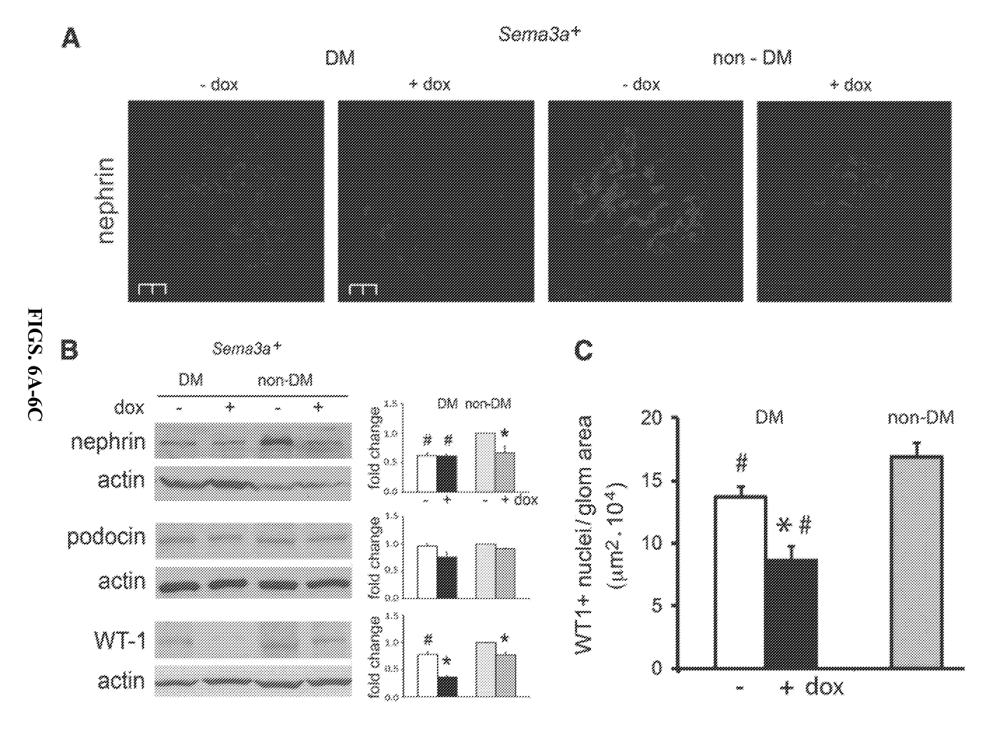

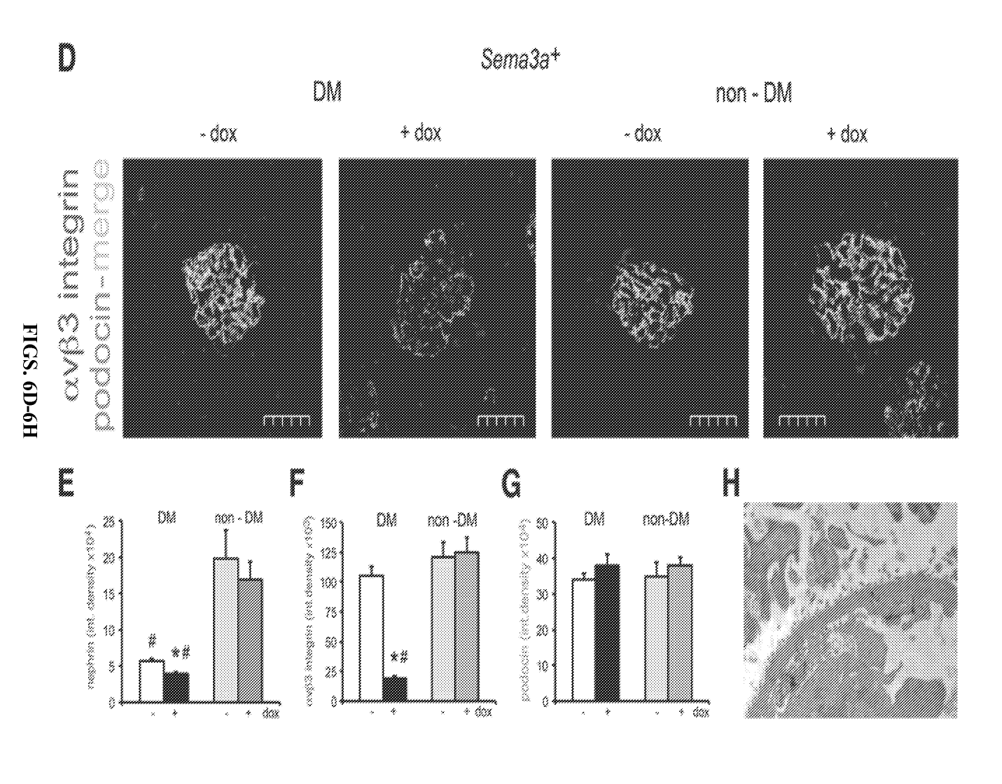

Nephrin downregulation was observed in all diabetic mice (FIGS. 6A-6B and FIG. 6E). Podocin was not significantly decreased by immunoblotting or immunofluorescence (FIGS. 6B and 6E), except in extensively damaged glomeruli (FIG. 6D). In addition, Sema3a⁺ gain-of-function induced a marked downregulation of WT1 not observed in control diabetic mice (FIG. 6B). Exacerbation of podocytopenia was confirmed by WT1⁺ cell count (FIG. 6C). Sema3a⁺ gain-of-function induced αvβ3 integrin downregulation in diabetic glomeruli (FIGS. 6D-6E), suggesting that decreased integrin activity may contribute to podocyte loss. Increased podocytopenia is consistent with the observation of focal GBM denudation in Sema3a⁺ gain-of-function diabetic glomeruli (FIG. 6H).

### Example 7: MICAL1 Mediates Sema3a-Induced Podocyte F-Actin Collapse

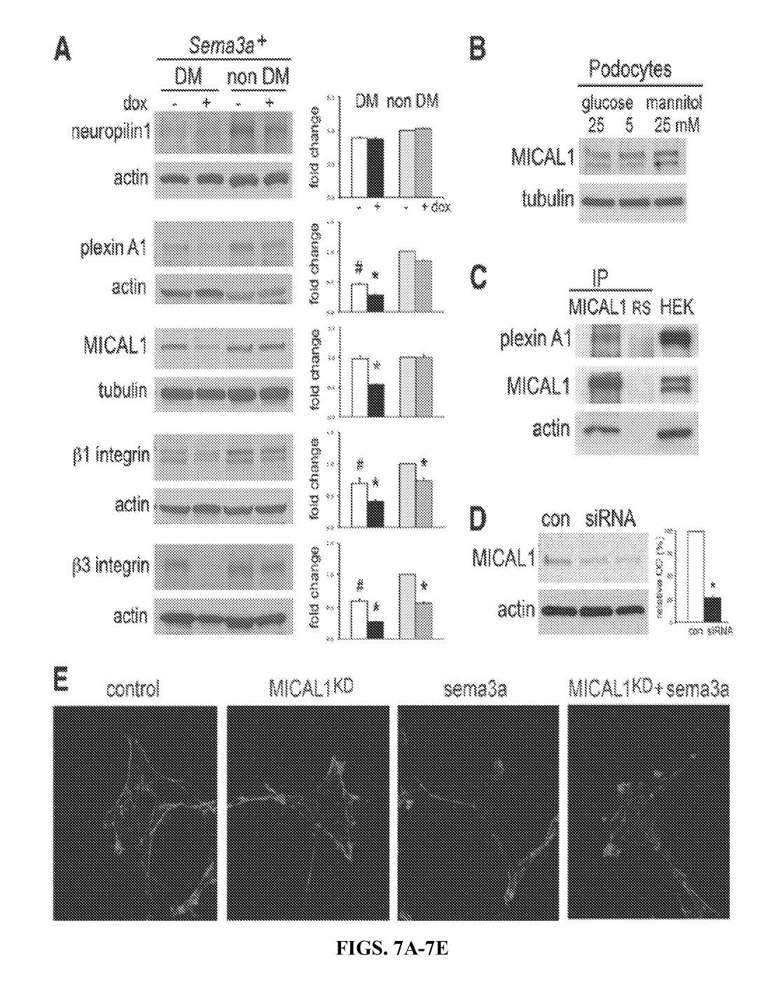

Sema3a signaling in the kidney downstream from plexinA₁-nephrin interaction was examined. Immunoblotting detected MICAL1 in whole kidney lysates and in cultured podocytes (FIGS. 7A-7B). PlexinA₁, MICAL1 and β3 integrin were significantly downregulated in Sema3a⁺ gain-of-function diabetic mice, whereas they were not disregulated in control diabetic or non-diabetic mice (FIG. 7A), suggesting that expression levels changes were due to Sema3a-induced severe diabetic nephropathy. Using co-immunoprecipitation, plexinA₁ was shown to interact with MICAL1 in cultured podocytes (FIG. 7C). Moreover, actin co-immunoprecipitates with the plexinA₁-MICAL1 complex (FIG. 7C). To evaluate whether Sema3a-induced podocyte F-actin collapse observed in vivo is due to MICAL1-mediated actin de-polymerization (Hung et al., Science 2011;334(6063):1710-1713), MICAL1 knockdown by siRNA in cultured podocytes were performed, which decreased MICAL1 expression by 73±2.6% (FIG. 7D). Using a cell assay and rhodamine phalloidin staining (Reidy et al., Am J Pathol 2013;183(4):1156-1168), MICAL1 knockdown was shown to render podocytes not susceptible to Sema3a-induced contraction (FIG. 7E). Together, these findings indicate that MICAL1 mediates Sema3a-induced podocyte F-actin collapse.

### Example 8: Xanthofulvin Prevents Sema3a-Induced Podocyte Damage and Attenuates Diabetic Nephropathy in Mice

Sema3a binding inhibitor xanthofulvin (Kumagai et al., J Antibiot (Tokyo) 2003;56(7):610-616; Axelrod et al., Angew Chem Int Ed Engl 2013;52(12):3421-3424) prevented Sema3a-induced podocyte F-actin collapse, shape change and contraction (FIGS. 8A-8B), as assessed by rhodamine-phalloidin and morphometry. Next, xanthofulvin infusion in vivo was tested for its capacity to ameliorate the phenotype of Sema3a⁺ gain-of-function diabetic mice. Xanthofulvin was administered by constant subcutaneous infusion (˜1.8 μg/d) for 30 days (weeks 8-12) with no apparent side effect, stable body weight and blood glucose (474±28 mg/dl, FIG. 11). Sema3a binding inhibition by xanthofulvin significantly decreases albuminuria, corrects hypoalbuminemia, abrogates renal insufficiency and markedly attenuates the diabetic glomerulosclerosis phenotype of Sema3a⁺ gain-of-function mice, as indicated by histology and TEM (FIG. 8C-E). Morphometric analysis revealed revealed significantly decreased GBM thickness and foot process effacement in xanthofulvin-treated Sema3a⁺ gain-of-function diabetic mice (FIG. 8F).

### Example 9: Deletion of PlexinA1 Attenuates Diabetic Nephropathy (DN) in Mice

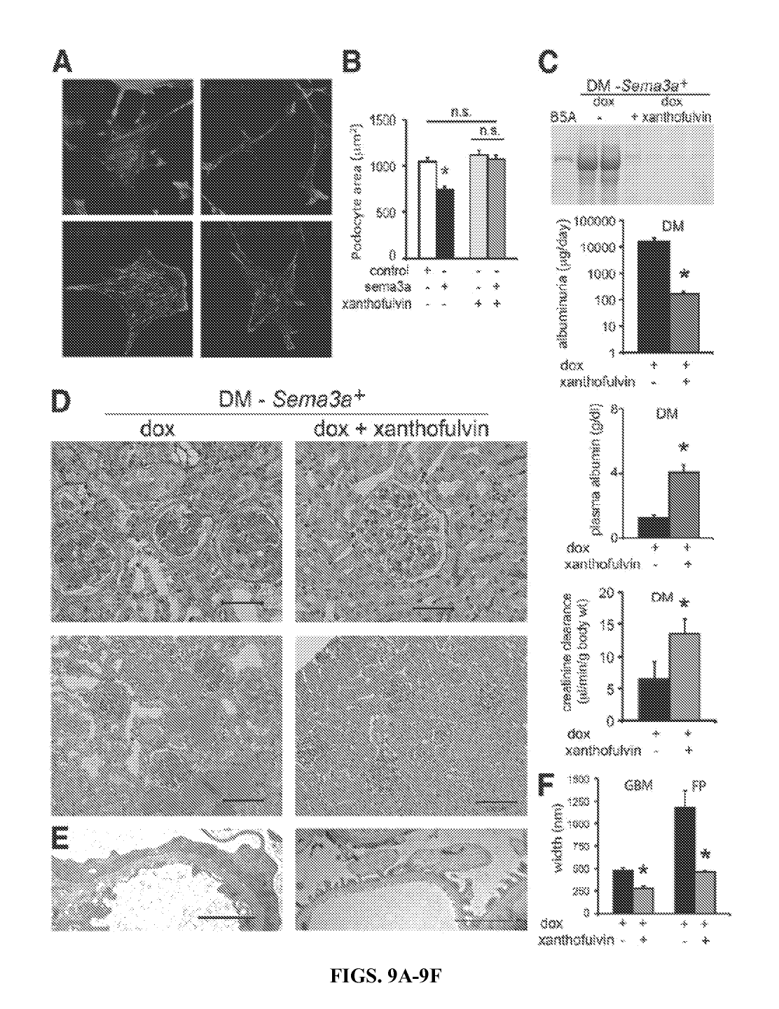

Mice carrying doxycycline regulated podocyte-specific plexinA1 deletion (plexinA1^{pod}) were generated to test whether Sema3a signaling in podocytes is responsible for the severe DN phenotype observed in diabetic Sema3a⁺ gain-of-function mice. In contrast to diabetic Sema3a⁺ gain-of-function mice (FIG. 9A, FIG. 9C and FIG. 9E), diabetic Sema3a⁺:plexinA1^{pod} showed mild mesangial proliferation and only focal foot process effacement (FIG. 9B, FIG. 9D, FIG. 9F and FIG. 9I), associated with mild albuminuria and normal creatinine clearance (FIGS. 9G-9H). Sema3a⁺:plexinA1^{pod} and Sema3a⁺ gain-of-function diabetic mice had similar hyperglycemia and severe polyuria (450±26 vs. 533±62 mg/dl, pNS, and FIG. 11). Collectively, Sema3a⁺:plexinA1^{pod} mice revealed a diabetic nephropathy phenotype indistinguishable from diabetic Sema3a⁺ gain-of-function treated with xanthofulvin or diabetic Sema3a⁺ control mice, demonstrating that deletion of podocyte Sema3a signaling attenuates diabetic nephropathy.

### Example 10

The present invention reveals that excess podocyte Sema3a promotes the development of advanced diabetic nephropathy and that SEMA3A localization to glomerular podocytes is increased in human advanced diabetic nephropathy. It is demonstrated herein that in diabetic mice podocyte Sema3a⁺ gain-of-function causes Kimmelstiel-Wilson-like nodular glomerulosclerosis, massive proteinuria and renal insufficiency. A signaling pathway that mediates Sema3a-induced podocyte F-actin collapse was identified by detecting plexinA₁ interaction with MICAL1 and actin in podocytes, demonstrating that MICAL1 is required to transduce Sema3a effect to the podocyte actin cytoskeleton. Sema3a inhibition by xanthofulvin was shown to abrogates Sema3a-induced podocyte contraction in vitro. Importantly, xanthofulvin-treatment or deletion of podocyte plexinA1 abrogate the diabetic nodular glomerulosclerosis resulting from Sema3a⁺ gain-of-function in vivo.

It is known in the art that VEGF-A and nitric oxide have essential roles in the pathogenesis of diabetic nephropathy (Tufro et al., Semin Nephro12012;32(4):385-393; Gnudi, Nephrol Dial Transplant. 2012;27(7):2642-2649; Tran et al., Annu Rev Cell Dev Biol 2007;23:263-292). Sema3a upregulation in diabetic mice was previously observed (Veron et al., Diabetologia 2011;54(5):1227-1241), and podocyte-specific Sema3a⁺ gain-of-function was shown to increase renal Sema3a ˜3-fold, leading to glomerular disease. It is evidenced herein podocyte SEMA3A, which alike VEGF-A is constitutively secreted by podocytes (Villegas et al., Mech Dev 2002;119 Suppl 1:S149-153), is significantly increased in renal biopsies from patients with class III and IV diabetic nephropathy, as compared with non-diabetic patients with hypertension, obesity or proteinuria due to minimal change disease. Although this finding does not elucidate the role of SEMA3A in the disease, it suggests that disregulation of semaphorin signaling might be relevant for human diabetic nephropathy. Increased tubular SEMA3A reported in renal biopsies from lupus nephritis patients (Vadasz et al., Arthritis Res Ther. 2012;14(3):R146), was not observed.

Podocyte Sema3a⁺ gain-of-function in diabetic mice was shown to induce accelerated and advanced diabetic nephropathy, as defined by the Animal Models of Diabetic Complications Consortium (AMDCC.org) and the Renal Pathology Society criteria (Tervaert et al., J Am Soc Nephrol 2010;21(4):556-563). Morphologically, diabetic mice with Sema3a⁺ gain-of-function developed mesangiolysis and nodular glomerulosclerosis in >50% of glomeruli, extensive mesangial sclerosis and interstitial fibrosis within 12-16 weeks. Moreover, Sema3a⁺ gain-of-function glomerular histology and TEM revealed multiple features of human advanced diabetic nephropathy (Nishi et al., Med Electron Microsc 2000; 33(2):65-73), including diffuse GBM thickening, widespread effacement and fusion of podocyte foot processes, marked podocytopenia, Kimmelstiel-Wilson-like nodular lesions, endothelial injury, fibrin caps and vascular pole hyalinosis. Few mouse models of diabetic nephropathy have been shown to develop diabetic nodular glomerulosclerosis, namely eNOS KO (Zhao et al., J Am Soc Nephrol 2006;17(10):2664-2669; Nakagawa et al., J Am Soc Nephrol 2007;18(2):539-550), β-cell calmodulin transgenics (Yuzawa et al., Diabetes. 2004; 53(12):3248-3257), podocyte-VEGF-A gain-of-function and BTBR Ob/Ob mice (Hudkins et al., J Am Soc Nephrol 2010;21(9):1533-1542). In most of these models, nodules become apparent late in the course of the disease (≧5 months), except podocyte-VEGF-A gain-of-function (Veron et al., Diabetologia 2011;54(5):1227-1241). Genetic background might contribute to these time-frame differences, as FVB mice are thought to be more susceptible to diabetic nephropathy than B6 mice (Brosius et al., J Am Soc Nephrol 2009;20(12): 2503-2512). Although Sema3a⁺ gain-of-function resulted in endothelial injury in both diabetic and non-diabetic kidneys, fibrin or other evidence of thrombotic microangiopathy was not observed, suggesting that the phenotype was due to severe diabetic nephropathy rather than overlap of diabetes and non-diabetic renal disease, as described in humans (Sharma et al., 2013;8(10):1718-1724). Genetic manipulation of Nos3, Vegf-a, Bkr1-2 and obesity in the setting of diabetes resulted in the most informative mouse models of advanced diabetic nephropathy (Zhao et al., J Am Soc Nephrol 2006;17(10):2664-2669; Nakagawa et al., J Am Soc Nephrol 2007;18(2):539-550; Kakoki et al., Proc Natl Acad Sci USA 2010;107(22):10190-10195), consistent with NOS3, and VEGFA polymorphisms linked to human diabetic nephropathy (Tufro et al., Semin Nephro12012;32(4):385-393; Gnudi, Nephrol Dial Transplant. 2012;27(7):2642-2649 ; Ezzidi et al., J Diabetes Complications 2008;22(5):331-338). Similarly, Sema3a⁺ gain-of-function diabetic mice develop the most advanced nodular glomerulosclerosis reported so far, mimicking class IV human diabetic nephropathy.

Functionally, Sema3a⁺ gain-of-function diabetic mice developed massive proteinuria leading to hypoalbuminemia and renal insufficiency, suggesting progressive diabetic nephropathy, consistent with the advanced morphologic phenotype. Previous mouse models of diabetic nodular glomerulosclerosis have shown some, but not all these functional abnormalities at once. For example, diabetic eNOS KO mice were shown to double their baseline BUN and increase albuminuria four-fold 5 months after the disease onset, β-cell calmodulin transgenic mice developed hyperfiltration, massive proteinuria and hypoalbuminemia at ≧6 months, podocyte-VEGF-A gain-of-function mice showed massive proteinuria and decreased hyperfiltration 3 months after disease onset (Veron et al., Diabetologia 2011;54(5):1227-1241), while BTBR Ob/Ob mice developed hyperfiltration and masssive albuminuria at 5 months of age (Hudkins et al., J Am Soc Nephrol 2010;21(9):1533-1542).

Normal Sema3a circulating levels in mice and humans are not well established. It is demontstrated herein that diabetes per se does not increase plasma Sema3a concentration in mice, as Sema3a was similar in control diabetic and non-diabetic mice. In contrast, podocyte Sema3a⁺ gain-of-function increases plasma Sema3a levels, and severe diabetic nephropathy appears to have an additive effect, probably due to decreased GFR, as suggested by plasma Sema3a inverse correlation with creatinine clearance in diabetic mice. Interestingly, the range of plasma Sema3a increase (˜3-fold) is similar to that of VEGF-A, reported in diabetic mice and humans (Veron et al., Diabetologia 2011;54(5):1227-1241 ; Hovind et al., Kidney Int Suppl 2000:75:S56-61Myocardial-specific Sema3a transgenic mice develop ventricular tachyarrhythmia and sudden death (Ieda et al., Nat Med 2007;13(5):604-612), unfortunately their plasma Sema3a level was not reported. Further studies will elucidate whether circulating Sema3a levels could be used as a biomarker of diabetic nephropathy, cardiovascular risk and/or disease progression.

Plasma VEGF-A is elevated in STZ-induced diabetes, irrespective of Sema3a transgene induction. Since local VEGF-A signaling at the glomerular filtration barrier, rather than higher circulating level, was shown to mediate advanced diabetic nephropathy (24), the measurement of kidney VEGF-A and VEGFR2, which were downregulated in Sema3a⁺ gain-of-function diabetic mice, indicated a decrease in VEGF-A signaling. Podocytopenia and Sema3a competition with VEGF-A for neuropilinl binding, abrogating local amplification of VEGFR2 signaling likely underlie these observations (Miao et al., J Cell Biol 1999;146(1):233-242). Together, these findings argue that the advanced diabetic nephropathy phenotype observed in Sema3a⁺ gain-of-function diabetic mice is not attributable to excess VEGF-A signaling. Previous studies have elucidated Sema3a effects on multiple cell types, and signaling pathways conserved from flies to humans (Tran et al., Annu Rev Cell Dev Biol 2007;23:263-292; Hung et al., Cytoskeleton (Hoboken) 2011;68(8):415-433). Sema3a repulsive cues lead to cell contraction (or retraction) by regulating actin dynamics (16,22,23,45). Landmark studies showed that Sema3a decreases motility and induces F-actin collapse in endothelial cells (19,45). It was demonstrated herein that both Sema3a receptors are expressed in podocytes, transducing cell autonomous Sema3a signals that induce podocyte contraction and apoptosis in vitro and in vivo (legas et al., Mech Dev 2002;119 Suppl 1:S149-153; Reidy et al., Development 2009;136(23):3979-3989; Reidy et al., Am J Pathol 2013;183(4):1156-1168; Guan et al., Kidney Int 2006;69(9): 1564-1569). Additional studies demonstrated that plexinA₁ interacts directly with nephrin (Reidy et al., Am J Pathol 2013;183(4):1156-1168). Here, for the first time MICAL1 protein in the kidney and in cultured podocytes were identified. It was also demonstrated that plexinAᵢ, MICAL1 and actin interact in podocytes. Upon Sema3a binding to neuropilin-plexinAᵢ complex, a direct interaction between MICAL1 C-terminus and plexinA₁ cytoplasmic domain releases MICAL1 autoinhibition and is required for Sema3a signaling (Terman et al., Cell 2002; 109(7):887-900, Schmidt et al., J Neurosci 2008;28(9):2287-2297). MICAL1 is a monoxygenase flavoprotein that selectively oxidizes actin Met46 and Met49 residues to disassemble F-actin in a reversible, redox-dependent manner (Hung et al., Nat Cell Biol 2013;15(12):1445-1454; Guan et al., Kidney Int 2006;69(9): 1564-1569). MICAL1 knockdown in cultured podocytes revealed that MICAL1 is required for Sema3a-induced podocyte shape changes and F-actin collapse, and suggest that Sema3a signaling may lead to H₂O₂ generation by MICAL1 in podocytes. This mechanism might be a critical contributor to Sema3a-induced podocyte and endothelial injury. The important role of reactive oxygen species in diabetic nephropathy is well established, although the beneficial effect of antioxidants on diabetic nephropathy is considered limited (Forbes et al., Physiol Rev 2013; 93(1):137-188; Tufro et al., Semin Nephrol 2012;32(4):385-393; Gnudi, Nephrol Dial Transplant. 2012;27(7):2642-2649). Identification and targeting of specific reactive oxygen species generating mechanisms, such as MICAL1, may unravel a novel therapeutic approach to diabetic nephropathy.

A key finding of the present invention is that xanthofulvin abrogates Sema3a-induced podocyte F-actin collapse in vitro and attenuates diabetic nephropathy in mice. Xanthofulvin is a specific Sema3a competitive binding inhibitor, naturally produced by Penicillium SPF-3059 and purified (Veron et al., J Am Soc Nephrol. 2014;25(8):1814-1824) or synthesized de novo (Axelrod et al., Angew Chem Int Ed Engl 2013;52(12):3421-3424). Both fungal and synthetic xanthofulvin prevent Sema3a-induced growth cone collapse in vitro (Axelrod et al., Angew Chem Int Ed Engl 2013;52(12):3421-3424; Kikuchi et al., J Biol Chem 2003; 278(44):42985-42991). Moreover, purified fungal xanthofulvin promotes functional recovery of injured spinal cord by decreasing apoptosis and enhancing angiogenesis in vivo (Lee et al., Mol Cell. 2013;51(3):397-404). Due to limited availability of fungal xanthofulvin, in vitro experiments were performed using synthetic xanthofulvin. It was determined herein that Sema3a inhibition by xanthofulvin has no deleterious effects in cultured podocytes. Most notably, in vivo xanthofulvin infusion administered to diabetic Sema3a⁺ gain-of-function mice decreased albuminuria, abrogated renal insufficiency and the diabetic nodular glomerulosclerosis phenotype, providing proof-of-principle that targeting Sema3a is beneficial in diabetic nephropathy.

To further confirm the relevant pathogenic role of increased Sema3a signaling in diabetic nephropathy podocyte plexinAl was deleted in Sema3a⁺ diabetic mice to abrogate Sema3a signaling. Notably, diabetic plexinA1 ^{pod}:Sema3a⁺ mice developed a mild diabetic nephropathy phenotype remarkably similar to that of xanthofulvin-treated and uninduced Sema3a⁺ diabetic mice. Together, these findings demonstrate that excess Sema3a signaling exacerbates diabetic nephropathy in mice.

Additional studies in other severe genetic type 1 and type 2 diabetic nephropathy models are needed to establish whether Sema3a signaling inhibition can prevent diabetic nephropathy or stop progression. Collectively, the human advanced diabetic nephropathy renal biopsies and mechanistic studies in diabetic mice presented herein suggest that establishing SEMA3A levels in healthy and diabetic individuals, their correlation with eGFR, proteinuria and glomerular immunoreactive SEMA3A in renal biopsies would advance the understanding of diabetic nephropathy. Sema3a is thought to function as an osteoprotective factor, negative regulator of immune response and angiogenesis, arrhythmogenic factor and potential biomarker of AKI (Ieda et al., Nat Med 2007;13(5):604-612; Kaneko et al., Nat Med 2006;12(12):1380-1389; Hayashi et al., Nature 2012; 485:69; Jayakumar et al., PLoS One 2013;8(3):e58446). Future studies targeting Sema3a signaling pathway should also evaluate these functions.

In summary, the present invention identifies podocyte SEMA3A upregulation in human biopsies with advanced diabetic nephropathy. Excess Sema3a plays a pathogenic role in diabetic nephropathy in mice leading to severe diabetic nodular glomerulosclerosis, massive proteinuria and renal failure, which can be attenuated by a Sema3a inhibitor or plexinA1 deletion. MICAL1 mediates Sema3a-plexinA₁ signals in podocytes leading to F-actin collapse. Collectively, these findings indicate that excess Sema3a promotes severe diabetic nephropathy and identify novel potential therapeutic targets.

The disclosures of each and every patent, patent application, and publication cited herein are hereby incorporated herein by reference in their entirety.

While the present invention has been disclosed with reference to specific embodiments, it is apparent that other embodiments and variations of the present invention may be devised by others skilled in the art without departing from the true spirit and scope of the invention. The appended claims are intended to be construed to include all such embodiments and equivalent variations.

1. A method for treating or preventing diabetic nephropathy in a mammal in need thereof, the method comprising administering to the mammal a therapeutically effective amount of an agent that inhibits podocyte Semaphorin3a (Sema3a).

2. (canceled)

3. A method for improving kidney function in a mammal having or at risk for having impaired kidney function, the method comprising administering to the mammal a therapeutically effective amount of an agent that inhibits podocyte Semaphorin3a (Sema3a).

4. (canceled)

5. (canceled)

6. (canceled)

7. (canceled)

8. The method of claim 1, wherein of the mammal has a type 1 or a type 2 diabetes.

9. The method of claim 1, wherein the administration route is at least one selected from the group consisting of parenteral and subcutaneous.

10. (canceled)

11. The method of claim 9, wherein the administration comprises at least one from the group consisting of at least one administration for at least 2 days, at least one administration for at least 20 days, at least one administration for at least 30 days, a continuous administration for at least 2 days, a continuous administration for at least 20 days, and a continuous administration for at least 30 days.

12. The method of claim 1, wherein the inhibiting agent comprises a specific Sema3a competitive binding inhibitor.

13. The method of claim 12, wherein the inhibitor comprises at least one from the group consisting of a natural product, a natural fungal product and a synthetic product.

14. The method of claim 13, wherein the inhibitor is xanthofulvin.

15. The method of claim 1, further comprising administering to the mammal an additional anti-diabetic therapeutic agent.

16. The method of claim 15, wherein the agent that inhibits podocyte Semaphorin3a (Sema3a) and the additional anti-diabetic therapeutic agent are co-administered to the mammal.

17. The method of claim 1, wherein the agent that inhibits podocyte Semaphorin3a (Sema3a) is in a pharmaceutical acceptable carrier.

18. A method for identifying or characterizing a compound useful for treating or preventing diabetic nephropathy in a mammal in need thereof, the method comprising measuring the level of expression or activity of at least one of podocyte Semaphorin3a (Sema3a) nucleic acid, Sema3a encoded polypeptide, Sema3a receptor and any combination thereof in a mammal treated with the compound and in an otherwise identical mammal not treated with the compound, wherein when the level of expression or activity of at least one of podocyte Sema3a nucleic acid, Sema3a encoded polypeptide, Sema3a receptor and any combination thereof is decreased in the treated mammal as compared with the untreated mammal, the compound is useful for treating or preventing diabetic nephropathy.

19. The method of claim 18, wherein the mammal is a human.

20. The method of claim 1, wherein the agent that inhibits podocyte Sema3a reduces at least one condition in the mammal selected from the group consisting of renal insufficiency, podocytopenia, glomerulosclerosis, glomerular filtration rate, proteinuria and albuminuria.

21. The method of claim 1, wherein the mammal is a human.

22. The method of claim 3, wherein of the mammal has a type 1 or a type 2 diabetes.

23. The method of claim 3, wherein the agent that inhibits podocyte Sema3a reduces at least one condition in the mammal selected from the group consisting of renal insufficiency, podocytopenia, glomerulosclerosis, glomerular filtration rate, proteinuria and albuminuria.

24. The method of claim 3, wherein the administration route is at least one selected from the group consisting of parenteral and subcutaneous.

25. The method of claim 24, wherein the administration comprises at least one from the group consisting of at least one administration for at least 2 days, at least one administration for at least 20 days, at least one administration for at least 30 days, a continuous administration for at least 2 days, a continuous administration for at least 20 days, and a continuous administration for at least 30 days.

26. The method of claim 3, wherein the inhibiting agent comprises a specific Sema3a competitive binding inhibitor.

27. The method of claim 26, wherein the inhibitor comprises at least one from the group consisting of a natural product, a natural fungal product and a synthetic product.

28. The method of claim 27, wherein the inhibitor is xanthofulvin.

29. The method of claim 3, further comprising administering to the mammal an additional anti-diabetic therapeutic agent.

30. The method of claim 29, wherein the agent that inhibits podocyte Semaphorin3a (Sema3a) and the additional anti-diabetic therapeutic agent are co-administered to the mammal.

31. The method of claim 3, wherein the agent that inhibits podocyte Semaphorin3a (Sema3a) is in a pharmaceutical acceptable carrier.

32. The method of claim 3, wherein the mammal is a human.


SEQUENCE LISTING (1 sequence)

SEQ ID NO: 1 - RNA, 21 nt, Mus musculus
caggugccaugacuaaguauu